(19) United States
(12) Patent Application Publication
(10) Pub. No.: US 2025/0241522 A1
(43) Pub. Date: Jul. 31, 2025

(54) PROCESSING DEVICE, ENDOSCOPE DEVICE, AND PROCESSING METHOD

(71) Applicant: FUJIFILM Corporation, Tokyo (JP)

(72) Inventors: Masaaki OOSAKE, Kanagawa (JP); Satoru TSUTO, Kanagawa (JP); Katsuyuki HIGA, Kanagawa (JP)

(73) Assignee: FUJIFILM Corporation, Tokyo (JP)

(21) Appl. No.: 19/182,520

(22) Filed: Apr. 17, 2025

Related U.S. Application Data
(63) Continuation of application No. PCT/JP2023/038538, filed on Oct. 25, 2023.

(30) Foreign Application Priority Data
Oct. 31, 2022 (JP) 2022-174967

(51) Int. Cl. A61B 1/00; A61B 1/31

(52) U.S. Cl. CPC A61B 1/00158; A61B 1/00055; A61B 1/31

(57) ABSTRACT

A processing device includes: a processor configured to: acquire a distance from a reference position on a movement path of an endoscope to a distal end of the endoscope that is moved along the movement path; acquire, from a memory, target position information indicating a target position on the movement path; and perform control of giving a notification based on the distance and the target position information.

### CROSS REFERENCE TO RELATED APPLICATION

This is a continuation of International Application No. PCT/JP2023/038538 filed on Oct. 25, 2023, and claims priority from Japanese Patent Application No. 2022-174967 filed on Oct. 31, 2022, the entire disclosures of which are incorporated herein by reference.

### BACKGROUND OF THE INVENTION

### 1. Field of the Invention

The present invention relates to a processing device, an endoscope device, and a processing method.

### 2. Description of the Related Art

Japanese Patent No. 3228622 discloses an endoscope system comprising an insertion length detection unit that detects an insertion distance of an insertion part of an endoscope into a body cavity, a recording unit that records an endoscopic image obtained via the endoscope and information on an insertion length detected by the insertion length detection unit on the same recording medium, an image reproduction/search unit that searches for image information corresponding to an insertion length closest to a current insertion length among endoscopic images recorded on the recording medium in the past based on information on the current insertion length detected by the insertion length detection unit and reproduces the searched image information, and a display unit that displays the image information reproduced by the image reproduction/search unit and a current endoscopic image at the same time.

### SUMMARY OF THE INVENTION

In the present disclosure, a technique capable of supporting endoscopy is provided.

A processing device according to an aspect of the present disclosure comprises: a processor configured to: acquire a distance from a reference position on a movement path of an endoscope to a distal end of the endoscope that is moved along the movement path; acquire target position information indicating a target position on the movement path from a memory; and perform control of giving a notification based on the distance and the target position information.

An endoscope device according to an aspect of the present disclosure comprises: the processing device and the endoscope.

A processing method according to an aspect of the present disclosure comprises: acquiring a distance from a reference position on a movement path of an endoscope to a distal end of the endoscope that is moved along the movement path; acquiring target position information indicating a target position on the movement path from a memory; and performing control of giving a notification based on the distance and the target position information.

According to the present disclosure, it is possible to provide a technique capable of supporting endoscopy.

### BRIEF DESCRIPTION OF THE DRAWINGS

FIG. 1 is a diagram illustrating a schematic configuration of an endoscope system 200.

FIG. 2 is a partial cross-sectional view illustrating a detailed configuration of a soft portion 10A of an endoscope 1.

FIG. 3 is a schematic diagram illustrating details of a magnetic pattern formed on a tubular member 17.

FIG. 4 is a schematic cross-sectional view taken along each of an A-A arrow and a B-B arrow in FIG. 3.

FIG. 5 is an exploded perspective view illustrating a configuration example of a detection unit 40.

FIG. 6 is a schematic diagram of a body part 42A of the detection unit 40 illustrated in FIG. 5 as viewed from a direction x.

FIG. 7 is a diagram illustrating an example of a position at which an insertion part 10 can be located in a through-hole 41.

FIG. 8 is a schematic diagram illustrating an example of a magnetic flux density detected by a magnetic detection unit 43.

FIG. 9 is a schematic diagram illustrating an example of a result of classifying the magnetic flux density illustrated in FIG. 8 according to magnitude thereof.

FIG. 10 is a schematic diagram illustrating another example of the result of classifying the magnetic flux density illustrated in FIG. 8 according to the magnitude thereof.

FIG. 11 is a schematic cross-sectional view illustrating a modification example of magnetic pole portions MAI and MA2 illustrated in FIG. 3 taken along the A-A arrow and the B-B arrow.

FIG. 12 is a diagram schematically illustrating a magnetic flux line generated in the magnetic pole portion MAI having the configuration illustrated in FIG. 11.

FIG. 13 is a schematic diagram illustrating a movement path of the insertion part 10 in an examination performed using the endoscope 1.

FIG. 14 is a graph illustrating a display example of examination data associated and recorded by a processor 8P.

FIG. 15 is a diagram illustrating an example of first table data.

### DESCRIPTION OF THE PREFERRED EMBODIMENTS

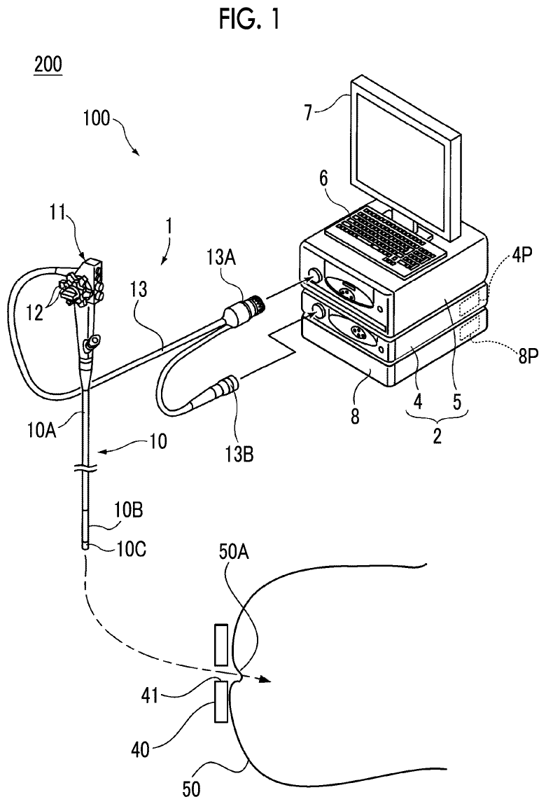

FIG. 1 is a diagram illustrating a schematic configuration of an endoscope system 200. The endoscope system 200 includes an endoscope device 100 having an endoscope 1 as an example of medical equipment that is used by being inserted into a body for examination, surgery, and the like, and a detection unit 40.

The endoscope 1 includes: an insertion part 10 which is an elongated instrument extending in one direction and is inserted into the body; an operating part 11 which is provided in a base end part of the insertion part 10 and is provided with an operation member for performing an observation mode switching operation, an imaging recording operation, a forceps operation, an air supply and water supply operation, a suction operation, an electric cautery operation, or the like; an angle knob 12 provided adjacent to the operating part 11; and a universal cord 13 including connector portions 13A and 13B that respectively connect the endoscope 1 to a light source device 5 and a processor device 4 in an attachable and detachable manner.

The operating part 11 is provided with a forceps port into which biopsy forceps as a treatment tool for collecting a biological tissue such as a cell or a polyp are inserted. It should be noted that, although the illustration is omitted in FIG. 1, various channels such as a forceps channel through which the biopsy forceps inserted from the forceps port are inserted, a channel for air supply and water supply, a channel for suction are provided inside the operating part 11 and the insertion part 10.

The insertion part 10 includes a soft portion 10A having flexibility, a bendable part 10B provided at a distal end of the soft portion 10A, and a distal end part 10C that is provided at a distal end of the bendable part 10B, and is harder than the soft portion 10A. An imaging element and an imaging optical system are built in the distal end part 10C.

The bendable part 10B is configured to be bendable by a rotational movement operation of the angle knob 12. Depending on a site or the like of a subject in which the endoscope 1 is used, the bendable part 10B can be bent in any direction and at any angle, and the distal end part 10C can be directed in a desired direction.

Hereinafter, a direction in which the insertion part 10 extends will be referred to as a longitudinal direction X. Further, one of radial directions of the insertion part 10 will be referred to as a radial direction Y. In addition, one of circumferential directions of the insertion part 10 (one of tangential directions of an outer peripheral edge of the insertion part 10) will be referred to as a circumferential direction Z. In the longitudinal direction X, a direction from a base end (operating part 11 side) of the endoscope 1 toward a distal end will be referred to as a longitudinal direction X1, and a direction from the distal end of the endoscope 1 to the base end will be referred to as a longitudinal direction X2. In addition, in the radial direction Y, one side will be referred to as a radial direction Y1, and the other side will be referred to as a radial direction Y2. The longitudinal direction X is one of directions different from the radial direction Y and the circumferential direction Z. The radial direction Y is one of directions different from the longitudinal direction X and the circumferential direction Z. In the present specification, the longitudinal direction X constitutes a first direction. Further, the radial direction Y constitutes a second direction intersecting the first direction. Further, the circumferential direction Z constitutes a third direction different from the first direction and the second direction.

In the example of FIG. 1, the insertion part 10 of the endoscope 1 is inserted into the body of a subject 50 from an anus 50A of the subject 50. The detection unit 40 has a rectangular plate shape as an example, and has a through-hole 41 into which the insertion part 10 can be inserted. The detection unit 40 is disposed between buttocks of the subject 50 and the insertion part 10 (that is, a movement path of the insertion part 10). The insertion part 10 reaches the anus 50A through the through-hole 41 of the detection unit 40, and is inserted into the body of the subject 50 from the anus 50A. In the present specification, the insertion part 10 constitutes an elongated instrument that is used by being relatively moved with respect to the detection unit 40.

The endoscope device 100 includes: the endoscope 1; a body part 2 consisting of the processor device 4 and the light source device 5 to which the endoscope 1 is connected; a display device 7 that displays a captured image and the like; an input unit 6 that is an interface for inputting various kinds of information to the processor device 4; and an expansion device 8 for expanding various functions.

The processor device 4 has various processors 4P that control the endoscope 1, the light source device 5, and the display device 7. The expansion device 8 has a processor 8P that performs various kinds of processing. Each of the processor 4P and the processor 8P is a central processing unit (CPU) as a general-purpose processor that executes software (a program including a display control program) to perform various functions, a programmable logic device (PLD) as a processor of which a circuit configuration can be changed after the manufacture, such as a field programmable gate array (FPGA), and a dedicated electric circuit as a processor having a circuit configuration specially designed for executing specific processing, such as an application specific integrated circuit (ASIC). Each of the processor 4P and the processor 8P may be composed of one processor, or composed of a combination of two or more processors of the same type or a different type (for example, a combination of a plurality of FPGAs or a combination of a CPU and an FPGA). More specifically, the hardware structure of each of the processor 4P and the processor 8P is an electric circuit (circuitry) in which circuit elements such as semiconductor elements are combined.

The expansion device 8 includes the processor 8P, a communication interface (an interface for communicating with the processor device 4 and the detection unit 40 to be described later) (not illustrated), and a memory composed of a recording medium such as a random access memory (RAM), a read only memory (ROM), a solid state drive (SSD), or a hard disk drive (HDD), and constitutes a processing device.

The processor 8P may perform lesion recognition processing of acquiring a captured image captured by the endoscope 1 from the processor device 4 and recognizing a lesion region in the captured image, treatment tool recognition processing of recognizing whether or not a treatment tool such as forceps or a needle is included in the captured image, and the like.

The lesion recognition processing refers to processing for performing detection of the lesion region from the captured image, and identification of the detected lesion region. In the lesion recognition processing, the processing for detecting the lesion region is referred to as detection processing, and the processing for identifying the lesion region is referred to as identification processing. The lesion recognition processing may be processing including at least the detection processing. The detection of the lesion region refers to finding a lesion region suspected of a lesion such as a malignant tumor or a benign tumor (lesion candidate region), from the captured image. The identification of the lesion region refers to identifying the type, nature, and the like of the detected lesion region, such as whether the lesion region detected by the detection processing is malignant or benign, what kind of disease in a case where the lesion region is malignant, or how much the degree of progress of the disease is. For example, both the lesion recognition processing and the treatment tool recognition processing can be executed by a recognition model generated by machine learning (for example, a neural network or a support vector machine) or image analysis on the captured image.

The various kinds of processing described below performed by the processor 8P may be performed by the processor 8P alone, or may be performed by being shared between the processor 8P and another processor. The other processor is, for example, a processor of a server in an examination system in which examination data generated by the endoscope system 200 is recorded, the processor 4P, or the like. Alternatively, various kinds of processing performed by the processor 8P can be performed by the processor 4P.

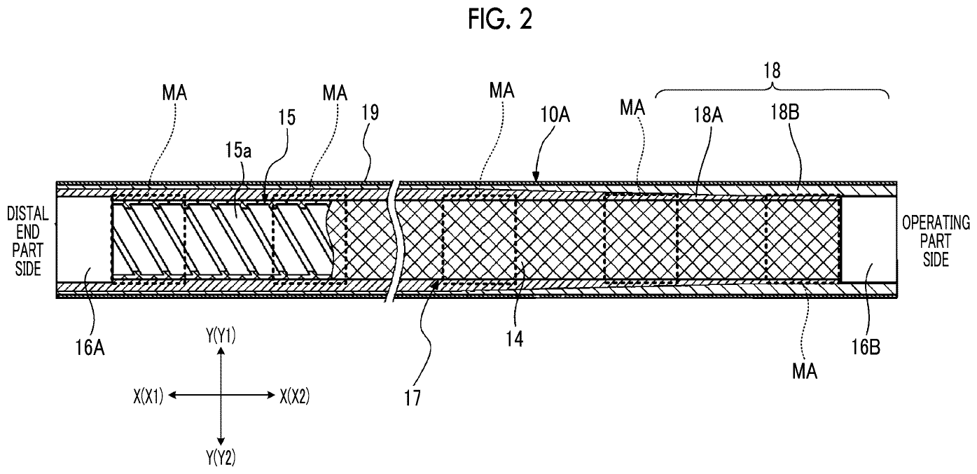

FIG. 2 is a partial cross-sectional view illustrating a detailed configuration of the soft portion 10A of the endoscope 1. The soft portion 10A, which forms most of a length of the insertion part 10, has flexibility over substantially the entire length thereof, and has a structure in which, in particular, a portion to be inserted into a body cavity or the like is rich in flexibility.

The soft portion 10A includes an outer skin layer 18 that constitutes a cylindrical member having an insulating property, and a tubular member 17 that is provided in the outer skin layer 18. The outer skin layer 18 is coated with a coating layer 19.

The tubular member 17 includes: a first member 14 that has a cylindrical shape, contains metal, and is covered with the outer skin layer 18; and a second member 15 that has a cylindrical shape, contains metal, and is inserted into the first member 14. In the example of FIG. 2, the second member 15 is composed of a spiral tube formed by spirally winding a metal strip 15a. Further, the first member 14 is composed of a cylindrical-shaped net body formed by braiding a metal wire. The first member 14 and the second member 15 that continuously extend in the longitudinal direction X and have a thin structure are formed by plastic processing, and the metal constituting these members includes austenitic stainless steel. The austenitic stainless steel cannot be magnetized in a state in which the plastic processing is not performed, but can be magnetized by performing the plastic processing. As described above, each of the first member 14 and the second member 15 constitutes a member that extends in the longitudinal direction X and contains metal.

The outer skin layer 18 is composed of, for example, a resin such as an elastomer, and has a multi-layer structure of an inner resin layer 18A and an outer resin layer 18B. The outer skin layer 18 may have a monolayer structure. In the first member 14 and the second member 15, a cap 16A is fitted to an end part on the distal end part 10C side, and a cap 16B is fitted to an end part on the operating part 11 side. The cap 16A and the cap 16B are covered with the outer skin layer 18. The soft portion 10A is connected to the bendable part 10B at the cap 16A, and is connected to the operating part 11 at the cap 16B.

The tubular member 17 of the soft portion 10A is formed with a magnetic pattern along the longitudinal direction X. The magnetic pattern along the longitudinal direction X refers to a pattern in which two types of magnetic pole regions, which are a negative pole (S pole) and a positive pole (N pole), are arranged in a predetermined arrangement pattern in the longitudinal direction X. As illustrated in FIG. 2, each of the first member 14 and the second member 15 is provided with a plurality of magnetic pole portions MA including the magnetic pole region. At least one of the two types of magnetic pole regions, which are the negative pole (S pole) and the positive pole (N pole), is formed on the magnetic pole portion MA. As described above, each of the first member 14 and the second member 15 constitutes the member that extends in the longitudinal direction X and has the magnetic pattern formed along the longitudinal direction X.

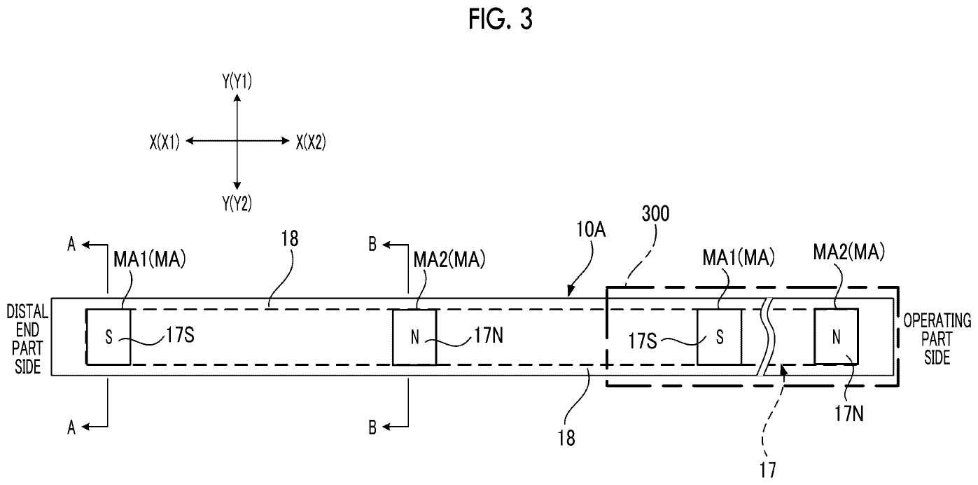

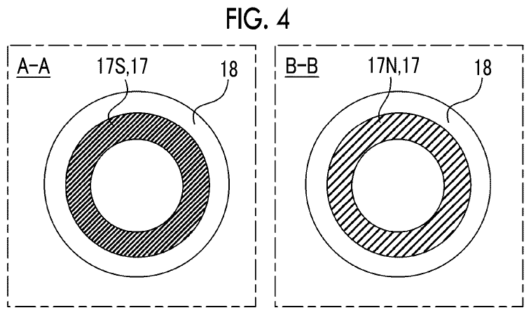

FIG. 3 is a schematic diagram illustrating details of the magnetic pattern formed on the tubular member 17. FIG. 4 is a schematic cross-sectional view taken along each of an A-A arrow and a B-B arrow in FIG. 3. As illustrated in FIGS. 3 and 4, in the tubular member 17, a magnetic pole portion MAI including a negative pole region 17S formed in an annular shape along the circumferential direction of the tubular member 17, and a magnetic pole portion MA2 including a positive pole region 17N formed in an annular shape along the circumferential direction of the tubular member 17 are provided to be alternately arranged in the longitudinal direction X. The total number of the magnetic pole portions MAI and the total number of the magnetic pole portions MA2 are the same.

Here, an example of a manufacturing method of the endoscope 1 including the tubular member 17 having the magnetic pattern illustrated in FIG. 3 will be described. First, the endoscope 1 having the configuration illustrated in FIG. 1 is manufactured by a well-known method. Next, a magnetic field generation device 300 is prepared, which has a cylindrical coil, and can generate a magnetic field in the cylindrical coil by allowing a current to flow through the cylindrical coil. Next, as illustrated in FIG. 3, the insertion part 10 of the endoscope 1 is inserted into the cylindrical coil of the magnetic field generation device 300 from the distal end side to relatively move the coil to a boundary portion between the operating part 11 and the soft portion 10A. In this state, a step of allowing an alternating current to flow through the cylindrical coil of the magnetic field generation device 300 to form a magnetic field, and pulling out the insertion part 10 from the cylindrical coil of the magnetic field generation device 300 in the longitudinal direction X2 at a constant speed is performed. In this step, a magnetic force of the tubular member 17 generated by the plastic processing is removed, and the tubular member 17 is demagnetized. In this step, it is preferable to pull out the insertion part 10 until the bendable part 10B and the distal end part 10C pass through the cylindrical coil, and to demagnetize the entire insertion part 10. That is, in the insertion part 10 of the endoscope 1, it is preferable that the bendable part 10B and the distal end part 10C are demagnetized. The demagnetization of a certain region means that a magnetic flux density detected from the region is equal to or less than the geomagnetism.

After the demagnetization of at least the tubular member 17 (soft portion 10A) is performed, a work of forming a state in which the cylindrical coil of the magnetic field generation device 300 is disposed on an outer periphery of the soft portion 10A at a predetermined position in the longitudinal direction X, and allowing the alternating current to flow through the cylindrical coil in that state to form the magnetic field is performed. By this work, the negative pole region 17S and the positive pole region 17N are formed over the entire circumferential direction of the tubular member 17 at positions in the vicinity of both ends of the cylindrical coil of the magnetic field generation device 300. Thereafter, by repeating this work while shifting the position of the soft portion 10A with respect to the cylindrical coil in the longitudinal direction X, the magnetic pattern illustrated in FIG. 3 can be formed on the tubular member 17.

By adopting such a manufacturing method, any magnetic pattern can be easily formed on the tubular member 17 of the soft portion 10A even in the endoscope 1 having the existing configuration or the endoscope 1 that has already been sold. In addition, by performing the demagnetization of the tubular member 17 of the soft portion 10A and then forming the magnetic pattern on the tubular member 17, the magnetic pattern having a desired magnetic force can be formed with high accuracy. Further, by forming the magnetic pole region by using the cylindrical coil, it is possible to form the magnetic pole region having a uniform magnetic force (magnetic flux density) over the entire outer periphery of the tubular member 17 in the magnetic pole portion MA. In FIG. 3, a boundary line between each of the negative pole region 17S and the positive pole region 17N, and the other region in the tubular member 17 is illustrated, but this boundary line is illustrated for convenience, and is invisible. It is preferable that information on the magnetic pattern formed on the tubular member 17 is recorded in a memory (for example, a memory provided in the expansion device 8) accessible by the processor 8P. The information on the magnetic pattern includes information indicating positions of the two types of magnetic pole regions in the tubular member 17, information indicating an arrangement pitch of the two types of magnetic pole regions in the tubular member 17, information indicating a range in which the magnetic pole region is formed on the insertion part 10, information indicating the position of the demagnetized region in the insertion part 10, or the like. The demagnetized region in the insertion part 10 constitutes an adjacent region adjacent to the region in which the magnetic pattern is formed on the insertion part 10. The bendable part 10B and the distal end part 10C are demagnetized regions in the insertion part 10, but the bendable part 10B and the distal end part 10C need only be configured to be distinguishable from the region in which the magnetic pattern is formed, and it is not essential that the bendable part 10B and the distal end part 10C are demagnetized. For example, magnetization may be performed with a pattern or a magnetic force that is clearly different from the magnetic pattern.

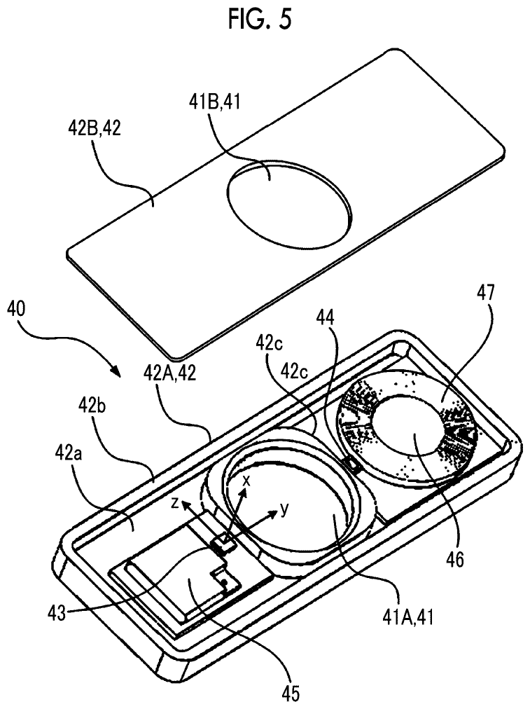

FIG. 5 is an exploded perspective view illustrating a configuration example of the detection unit 40. The detection unit 40 includes a housing 42 having the through-hole 41; and a magnetic detection unit 43, a magnetic detection unit 44, a communication chip 45, a storage battery 46, and a power receiving coil 47 that are accommodated in the housing 42.

The housing 42 includes: a body part 42A including a flat plate portion 42a that has a rectangular flat plate shape and has a through-hole 41A penetrating in a thickness direction, a side wall portion 42b that has a rectangular frame shape of rising from an outer peripheral edge portion of the flat plate portion 42a in the thickness direction of the flat plate portion 42a, and an inner wall portion 42c that has a cylindrical shape of rising from a peripheral edge portion of the through-hole 41A in the flat plate portion 42a in the thickness direction of the flat plate portion 42a; and a lid portion 42B that has a rectangular flat plate shape for closing an accommodation space surrounded by the flat plate portion 42a, the side wall portion 42b, and the inner wall portion 42c. The magnetic detection unit 43, the magnetic detection unit 44, the communication chip 45, the storage battery 46, and the power receiving coil 47 are accommodated in this accommodation space.

A through-hole 41B penetrating in the thickness direction is formed on the lid portion 42B, and in a state in which the lid portion 42B closes the accommodation space, the through-hole 41A and the through-hole 41B communicate with each other through an inner peripheral portion of the inner wall portion 42c to form the through-hole 41 into which the endoscope 1 can be inserted. It is preferable that the through-hole 41 has a perfect circular shape as viewed from an axial direction of the inner wall portion 42c (direction in which the endoscope 1 is inserted). The housing 42 is preferably composed of a resin or the like in order to reduce the weight and the cost, and preferably has a structure that prevents moisture from entering the accommodation space.

Each of the magnetic detection unit 43 and the magnetic detection unit 44 is disposed close to the inner wall portion 42c, and is a three-axis magnetic sensor that can detect a magnetic flux density in a direction x (direction along the axis of the through-hole 41) along the axis of the inner wall portion 42c, a magnetic flux density in a radial direction y of the through-hole 41, and a magnetic flux density in a direction z orthogonal to the direction x and the radial direction y.

In a state in which the insertion part 10 of the endoscope 1 is inserted into the through-hole 41, the longitudinal direction X of the insertion part 10 and the direction x match each other, the radial direction Y of the insertion part 10 and the radial direction y match each other, and the circumferential direction Z of the insertion part 10 and the direction z match each other. Therefore, each of the magnetic detection unit 43 and the magnetic detection unit 44 is configured to detect a magnetic flux density BX in the longitudinal direction X of the insertion part 10, a magnetic flux density BY in the radial direction Y of the insertion part 10, and a magnetic flux density BZ in the circumferential direction Z of the insertion part 10. Each of the magnetic detection unit 43 and the magnetic detection unit 44 may include three magnetic sensors, which are a uniaxial magnetic sensor that can detect the magnetic flux density BX, a uniaxial magnetic sensor that can detect the magnetic flux density BY, and a uniaxial magnetic sensor that can detect the magnetic flux density BZ. In the present specification, the magnetic flux density BX constitutes a first magnetic flux density, the magnetic flux density BY constitutes a second magnetic flux density, and the magnetic flux density BZ constitutes a third magnetic flux density.

Each of the magnetic detection unit 43 and the magnetic detection unit 44 need only be able to detect the magnetic flux density including a component in the longitudinal direction X, the magnetic flux density including a component in the radial direction Y, and the magnetic flux density including a component in the circumferential direction Z, and three detection axis directions may not exactly match the longitudinal direction X, the radial direction Y, and the circumferential direction Z, respectively. In the magnetic sensor, in a case in which a first detection axis direction is different from the radial direction Y and the circumferential direction Z, a second detection axis direction is different from the longitudinal direction X and the circumferential direction Z, and a third detection axis direction is different from the radial direction Y and the longitudinal direction X, the magnetic sensor can detect the magnetic flux density including the component in the longitudinal direction X, can detect the magnetic flux density including the component in the radial direction Y, and can detect the magnetic flux density including the component in the circumferential direction Z.

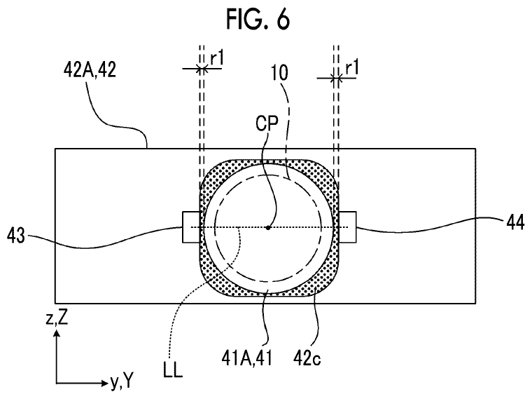

FIG. 6 is a schematic diagram of the body part 42A of the detection unit 40 illustrated in FIG. 5 as viewed from the direction x. As illustrated in FIG. 6, the magnetic detection unit 43 and the magnetic detection unit 44 are disposed at positions facing each other with a center CP of the through-hole 41 interposed therebetween as viewed in the direction x. That is, in a state of being viewed in the direction x, a midpoint of a line segment LL connecting the magnetic detection unit 43 and the magnetic detection unit 44 substantially matches the center CP of the through-hole 41. In other words, a distance from the magnetic detection unit 43 to the center CP of the through-hole 41 and a distance from the magnetic detection unit 44 to the center CP of the through-hole 41 substantially match each other.

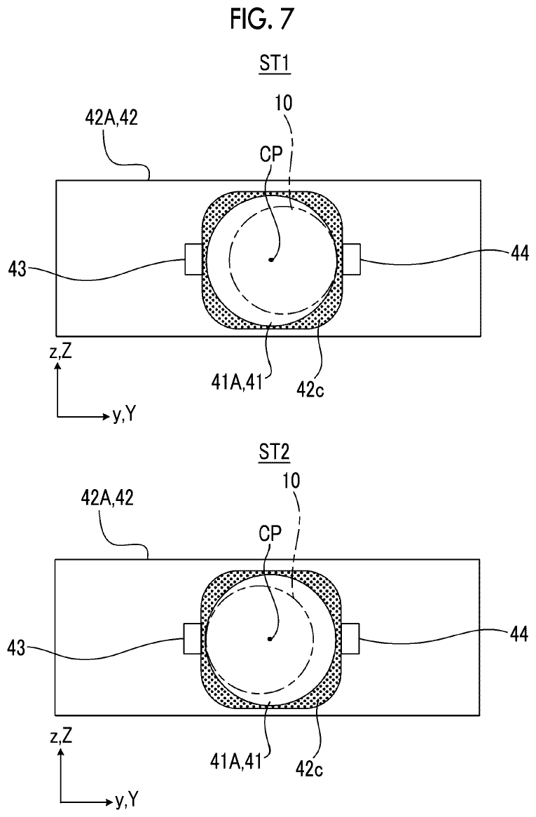

FIG. 7 is a diagram illustrating an example of a position at which the insertion part 10 can be located in the through-hole 41. A state STI of FIG. 7 illustrates a state in which the insertion part 10 is most distant from the magnetic detection unit 43 in the radial direction Y in the through-hole 41. A state ST2 of FIG. 7 illustrates a state in which the insertion part 10 is most distant from the magnetic detection unit 44 in the radial direction Y in the through-hole 41. A detection range and an installation position of each of the magnetic detection unit 43 and the magnetic detection unit 44 are determined such that the magnetic flux density can be detected with high accuracy from the magnetic pattern formed on the tubular member 17 in any of the state STI and the state ST2 of FIG. 7.

In the present embodiment, as illustrated in FIG. 6, a thickness of a portion of the inner wall portion 42c, the portion being at the same position as the center CP in the direction z, is a thickness r1. The thickness rl is 0.5 mm, for example. In a case in which the magnetic force of the magnetic pole region formed on the tubular member 17 is defined by the magnetic flux density detected at a position distant from an outer surface of the insertion part 10 in the radial direction of the insertion part 10 by 0.5 mm, it is preferable that the magnetic force has a value that is sufficiently larger than the geomagnetism and is equal to or larger than a value (specifically, 500 microtesla) suitable for the performance of a general magnetic sensor. In addition, for example, in the state STI or the state ST2 of FIG. 7, it is more preferable that the magnetic force of the magnetic pole region formed on the tubular member 17 is in a range of 1000 microtesla to 1500 microtesla such that the magnetic detection unit 43 and the magnetic detection unit 44 can detect the magnetic flux density with high accuracy. However, it is preferable that an upper limit value of the magnetic force of the magnetic pole region formed on the tubular member 17 is equal to or less than 20 millitesla such that the insertion part 10 does not adhere to another metal. In consideration of the maximum sensitivity of the general magnetic sensor, it is more preferable that the upper limit value of the magnetic force of the magnetic pole region formed on the tubular member 17 is equal to or less than 2 millitesla.

As illustrated in FIG. 7, the position of the insertion part 10 in the through-hole 41 can be changed. However, by obtaining the arithmetic mean of the magnetic flux density BX detected from the tubular member 17 by the magnetic detection unit 43 and the magnetic flux density BX detected from the tubular member 17 by the magnetic detection unit 44, it is possible to detect the magnetic flux density BX according to the magnetic pattern regardless of the position of the insertion part 10 in the through-hole 41. Similarly, by obtaining the arithmetic mean of the magnetic flux density BY detected from the tubular member 17 by the magnetic detection unit 43 and the magnetic flux density BY detected from the tubular member 17 by the magnetic detection unit 44, it is possible to detect the magnetic flux density BY according to the magnetic pattern regardless of the position of the insertion part 10 in the through-hole 41. Similarly, by obtaining the arithmetic mean of the magnetic flux density BZ detected from the tubular member 17 by the magnetic detection unit 43 and the magnetic flux density BZ detected from the tubular member 17 by the magnetic detection unit 44, it is possible to detect the magnetic flux density BZ according to the magnetic pattern regardless of the position of the insertion part 10 in the through-hole 41.

The communication chip 45 illustrated in FIG. 5 transmits information on the magnetic flux density detected by each of the magnetic detection unit 43 and the magnetic detection unit 44 to the expansion device 8 by wireless communication. In the present specification, the communication chip 45 constitutes an output unit that outputs the information detected by the magnetic detection unit 43 and the magnetic detection unit 44 to the outside. This information on the magnetic flux density may be transmitted to the processor device 4, and in this case, this information is transmitted by the processor 4P to the processor 8P of the expansion device 8.

The storage battery 46 is charged by the power received by the power receiving coil 47 by the noncontact power supply. The magnetic detection unit 43, the magnetic detection unit 44, and the communication chip 45 are operated by the power supplied from the storage battery 46. The detection unit 40 has a start-up switch (not illustrated). By performing an operation to turn on the start-up switch, the power supply from the storage battery 46 to the magnetic detection unit 43, the magnetic detection unit 44, and the communication chip 45 is started. The detection unit 40 may have a configuration in which the start-up switch is not provided and the power supply to the magnetic detection unit 43, the magnetic detection unit 44, and the communication chip 45 is started by receiving wireless power supply from the outside. In a case in which the start-up switch is not provided, a structure in which the accommodation space of the housing 42 is completely scaled can be easily realized.

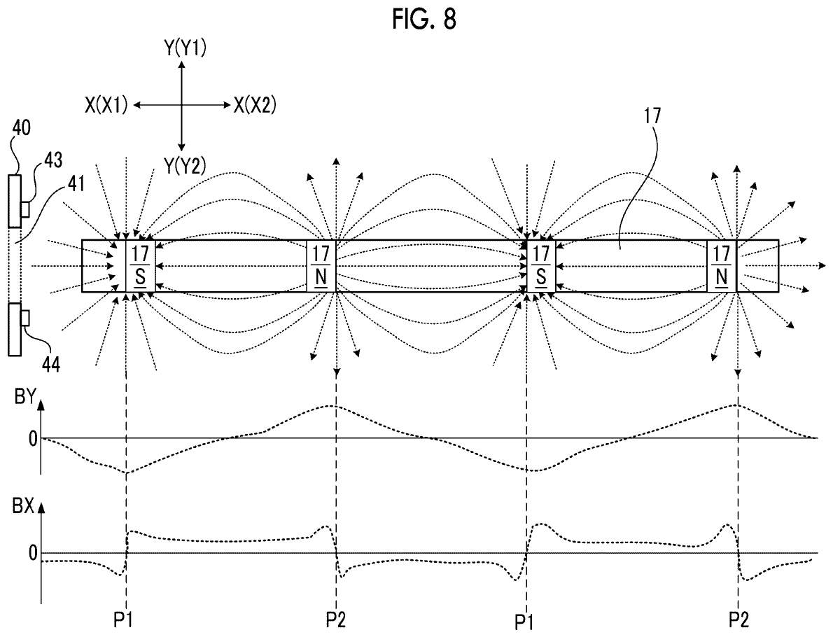

FIG. 8 is a schematic diagram illustrating an example of the magnetic flux density detected by the magnetic detection unit 43. Since the magnetic flux density detected by the magnetic detection unit 44 is the same as that in FIG. 8, the illustration is omitted. Two graphs illustrated in FIG. 8 illustrate the magnetic flux density BX and the magnetic flux density BY that are detected by the magnetic detection unit 43 in a case where the soft portion 10A is moved in the longitudinal direction X1 through the through-hole 41. In FIG. 8, a magnetic flux line from the positive pole region 17N to the negative pole region 17S adjacent to the positive pole region 17N in the longitudinal direction X is indicated by a broken line arrow.

In a case in which the soft portion 10A (tubular member 17) is moved toward the through-hole 41 of the detection unit 40 illustrated in the upper left of FIG. 8, as illustrated in the graph of FIG. 8, the magnetic flux density BX detected by the magnetic detection unit 43 has a positive value between each positive pole region 17N and the negative pole region 17S adjacent to the positive pole region 17N in the longitudinal direction X1, and has a negative value between each positive pole region 17N and the negative pole region 17S adjacent to the positive pole region 17N in the longitudinal direction X2. In addition, the magnetic flux density BY detected by the magnetic detection unit 43 has a negative value and a large absolute value in the vicinity of the negative pole region 17S, has a positive value and a large absolute value in the vicinity of the positive pole region 17N, and has a value close to zero in the vicinity of an intermediate position between the negative pole region 17S and the positive pole region 17N.

Regarding the magnetic flux densities detected from the magnetic pattern formed on the tubular member 17 at a plurality of positions in the longitudinal direction X of the tubular member 17, each of the magnetic flux density BX and the magnetic flux density BY is periodically changed with positive and negative values, and the phases of the magnetic flux density BX and the magnetic flux density BY are shifted from each other in the longitudinal direction X. In the negative pole region 17S, an end (portion of a position P1 in FIG. 8) in the longitudinal direction X where the absolute value of the magnetic flux density BY is the maximum value is hereinafter referred to as a negative pole end. In the positive pole region 17N, an end (portion of a position P2 in FIG. 8) in the longitudinal direction X where the absolute value of the magnetic flux density BY is the maximum value is hereinafter referred to as a positive pole end.

As an example, by magnetizing the tubular member 17 using the method described above by setting a length of the cylindrical coil of the magnetic field generation device 300 in the axial direction to 60 mm, an inner diameter of the cylindrical coil of the magnetic field generation device 300 to 18 mm, and a movement pitch of the cylindrical coil in the longitudinal direction X to 144 mm, it is possible to form the magnetic pattern in which a distance between the negative pole end and the positive pole end is 72 mm. In the example of FIG. 8, for example, by disposing the cylindrical coil between the negative pole region 17S at the left end and the positive pole region 17N adjacent to the right side of the negative pole region 17S to form the magnetic field, it is possible to form these two magnetic pole regions. Then, from that state, by relatively moving the cylindrical coil by 144 mm in the longitudinal direction X2 to form the magnetic field in that state, it is possible to form the positive pole region 17N at the right end and the negative pole region 17S adjacent to the left side of the positive pole region 17N. In this manner, it is possible to form the magnetic pattern in which the distance (distance between the position P1 and the position P2) between the positive pole end and the negative pole end which are alternately formed in the longitudinal direction X is 72 mm.

In the endoscope system 200, the processor 8P of the expansion device 8 acquires the information on the magnetic flux densities detected by the magnetic detection unit 43 and the magnetic detection unit 44, from the detection unit 40, and determines the movement state of the insertion part 10 in the longitudinal direction X on the basis of the acquired magnetic flux density BX and magnetic flux density BY. The movement state of the insertion part 10 determined here includes: a movement direction indicating in which direction in the longitudinal direction X the insertion part 10 is moved with respect to the detection unit 40; and a movement amount (movement distance) indicating how much distance the insertion part 10 inserted into the through-hole 41 of the detection unit 40 has moved in the longitudinal direction X with respect to the detection unit 40. The processor 8P obtains the arithmetic mean of the magnetic flux densities BX respectively detected at the same timing by the magnetic detection unit 43 and the magnetic detection unit 44, obtains the arithmetic mean of the magnetic flux densities BY respectively detected at the same timing by the magnetic detection unit 43 and the magnetic detection unit 44, and determines the movement state of the insertion part 10 on the basis of the magnetic flux density BX and the magnetic flux density BY obtained by these arithmetic means.

The processor 8P classifies the magnetic flux density BX into a plurality of pieces of information according to the magnitude thereof, classifies the magnetic flux density BY into a plurality of pieces of information according to the magnitude thereof, and determines the movement state of the insertion part 10 in the longitudinal direction X on the basis of a combination of any of the plurality of pieces of information obtained by classifying the magnetic flux density BX and any of the plurality of pieces of information obtained by classifying the magnetic flux density BY.

Specifically, the processor 8P sets a first threshold value th (for example, “0”) as a threshold value for classifying the magnetic flux density BX into two levels, and sets a second threshold value th1 (positive value larger than 0) and a second threshold value th2 (negative value less than 0) as a threshold value for classifying the magnetic flux density BY into three levels. Moreover, the processor 8P classifies the magnetic flux density BX by setting a value larger than the first threshold value th as a high level H and setting a value less than the first threshold value th as a low level L. Further, the processor 8P classifies the magnetic flux density BY by setting a value larger than the second threshold value th1 as the high level H, setting a value between the second threshold value th1 and the second threshold value th2 as a middle level M, and setting a value less than the second threshold value th2 as the low level L. The result of classifying the magnetic flux density BX in this manner is also referred to as a classification level of the magnetic flux density BX, and the result of classifying the magnetic flux density BY in this manner is also referred to as a classification level of the magnetic flux density BY. In the present specification, among the classification levels of the magnetic flux density BX, the high level constitutes one of fourth information and fifth information, and the low level constitutes the other of the fourth information and the fifth information. In addition, among the classification levels of the magnetic flux density BY, the high level constitutes one of first information and second information, the low level constitutes the other of the first information and the second information, and the middle level constitutes third information.

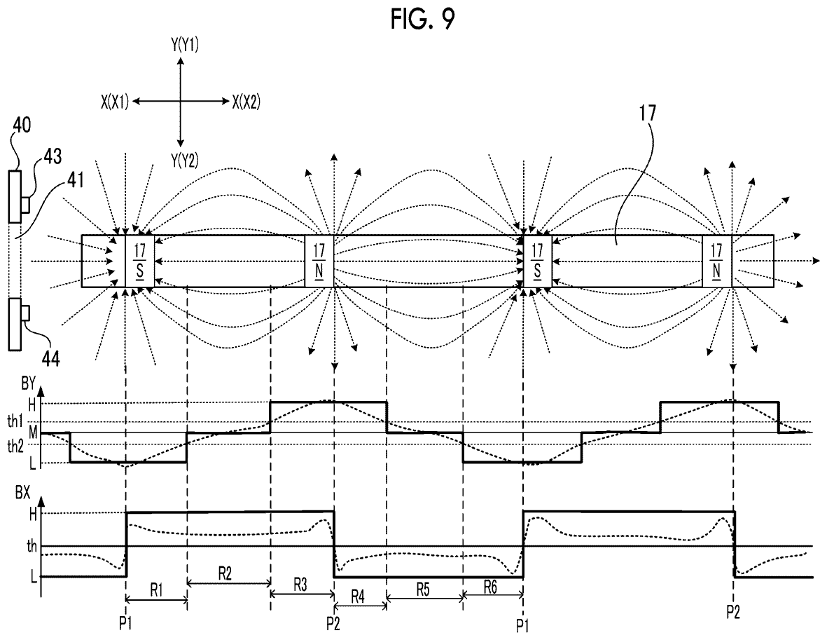

In FIG. 9, the result (classification level) of classifying the magnetic flux density BX and the magnetic flux density BY in the graphs illustrated in FIG. 8 is indicated by a thick solid line. As illustrated in FIG. 9, in the tubular member 17, a range between two adjacent positions PI (between the negative pole ends) is divided into: a region R1 in which the magnetic flux density BX is at the high level and the magnetic flux density BY is at the low level; a region R2 in which the magnetic flux density BX is at the high level and the magnetic flux density BY is at the middle level; a region R3 in which the magnetic flux density BX is at the high level and the magnetic flux density BY is at the high level; a region R4 in which the magnetic flux density BX is at the low level and the magnetic flux density BY is at the high level; a region R5 in which the magnetic flux density BX is at the low level and the magnetic flux density BY is at the middle level; and a region R6 in which the magnetic flux density BX is at the low level and the magnetic flux density BY is at the low level. As described above, the range between the negative pole ends adjacent to each other in the longitudinal direction X can be divided into six regions R1 to R6 depending on the combination of the classification level of the magnetic flux density BX and the classification level of the magnetic flux density BY.

By monitoring the thick solid lines (classification levels of the magnetic flux densities BX and BY) illustrated in FIG. 9, the processor 8P determines the movement direction of the insertion part 10 with respect to the detection unit 40, and the movement amount (movement distance) of the insertion part 10 in the longitudinal direction X starting from the position of the detection unit 40.

For example, in a case in which the negative pole region 17S provided on the most distal end side of the tubular member 17 passes through the through-hole 41, the processor 8P detects that the region R1 at the most distal end of the tubular member 17 is located in the through-hole 41, from the combination of the classification level of the magnetic flux density BX and the classification level of the magnetic flux density BY, and detects the position as a reference position. The distance (referred to as a distance L1) in the longitudinal direction X from the negative pole region 17S provided on the most distal end side of the tubular member 17 to the distal end of the distal end part 10C is known. Therefore, in a case in which this reference position is detected, the processor 8P determines that the movement distance of the insertion part 10 with respect to the detection unit 40 is “0”, and further determines that an insertion length (distance from the reference position (through-hole 41) to the distal end of the insertion part 10) of the insertion part 10 into the body of the subject 50 is the distance L1.

After the reference position is detected, in a case in which it is determined that the region of the tubular member 17 passing through the through-hole 41 is being changed in a direction from the region R1 to the region R6 according to the classification levels of the magnetic flux densities BX and BY, the processor 8P determines that the insertion part 10 is being moved in the longitudinal direction X1. In addition, in a case in which it is determined that the insertion part 10 is being moved in the longitudinal direction X1, the processor 8P increases the movement distance of the insertion part 10 in the longitudinal direction X1 by a unit distance ΔL and increases the insertion length of the insertion part 10 into the body of the subject 50 by the unit distance ΔL, each time the region of the tubular member 17 passing through the through-hole 41 is changed by one (for example, a change from the region R1 to the region R2 or a change from the region R2 to the region R3). The unit distance ΔL can be a value obtained by dividing an interval between the adjacent negative pole regions 17S by 6.

On the other hand, in a case in which it is determined that the region of the tubular member 17 passing through the through-hole 41 is being changed in a direction from the region R6 to the region R1 according to the classification levels of the magnetic flux densities BX and BY, the processor 8P determines that the insertion part 10 is being moved in the longitudinal direction X2. In addition, in a case in which it is determined that the insertion part 10 is being moved in the longitudinal direction X2, the processor 8P decreases the movement distance of the insertion part 10 in the longitudinal direction X1 by a unit distance ΔL and decreases the insertion length of the insertion part 10 into the body of the subject 50 by the unit distance ΔL, each time the region of the tubular member 17 passing through the through-hole 41 is changed by one.

Depending on the movement speed of the insertion part 10, it can also be determined that the region of the tubular member 17 passing through the through-hole 41 is changed from the region R1 to the region R3 or is changed from the region R3 to the region R1. In a case in which it is determined that the region of the tubular member 17 passing through the through-hole 41 is changed by two in this manner, the processor 8P need only increase or decrease the insertion length of the insertion part 10 by twice the unit distance ΔL.

The processor 8P displays the information on the insertion length determined in this manner on the display device 7, outputs the information by voice from a speaker (not illustrated), or transmits the information to an operator of the endoscope 1 by vibration of a vibrator provided in the operating part 11. As a result, it is possible to accurately record an imaging position by the endoscope 1, guide or evaluate the operation of the endoscope 1, and the like.

As described above, by demagnetizing the distal end part 10C and the bendable part 10B in the insertion part 10, the processor 8P can easily detect the reference position. Specifically, in a case in which the insertion part 10 is inserted into the through-hole 41 from the distal end side and is moved in the longitudinal direction X1, both the magnetic flux density BX and the magnetic flux density BY are values in the vicinity of “0” while the distal end part 10C and the bendable part 10B pass through the through-hole 41. Further, at a point in time at which the negative pole region 17S on the most distal end side of the tubular member 17 reaches the through-hole 41, the magnetic flux density BX and the magnetic flux density BY are a combination of the high level and the low level as illustrated in FIG. 9, and therefore, it is possible to easily detect the reference position by the fluctuation of the magnetic flux density.

As described above, the processor 8P classifies the magnetic flux density BX into two of the high level and the low level, classifies the magnetic flux density BY into three of the high level, the middle level, and the low level, and determines the movement state of the insertion part 10 in the longitudinal direction X on the basis of the combination thereof. In this way, by monitoring the change in the combination of the classification level of the magnetic flux density BX and the classification level of the magnetic flux density BY, the movement direction, the movement distance, and the insertion length of the insertion part 10 can be determined. With the endoscope system 200, such an effect can be realized only by magnetizing the endoscope 1 having a general-purpose configuration and adding the detection unit 40, so that a construction cost of the system can be reduced. In addition, since the movement direction, the movement distance, and the insertion length of the insertion part 10 are determined on the basis of the information on the magnetic flux density that can be acquired non-optically, even in a case in which the insertion part 10 is dirty, the determination accuracy is not reduced, and thus it is practical.

In addition, by using the combination of the classification level of the magnetic flux density BX and the classification level of the magnetic flux density BY, it is possible to determine the movement distance of the insertion part 10 with a resolution (for example, a unit of ⅓ of the interval) finer than the interval between the two types of adjacent magnetic pole regions (negative pole region 17S and positive pole region 17N). In this way, the movement distance can be finely determined, which can be useful for accurate recording of the imaging position by the endoscope 1, guide or evaluation of the operation of the endoscope 1, and the like.

In addition, the processor 8P obtains the arithmetic mean of the magnetic flux density detected by the magnetic detection unit 43 and the magnetic flux density detected by the magnetic detection unit 44, and determines the movement direction, the movement distance, and the insertion length of the insertion part 10 on the basis of the magnetic flux density of the arithmetic mean. Therefore, it is possible to obtain the change in the magnetic flux density according to the magnetic pattern regardless of the position of the insertion part 10 in the through-hole 41. In addition, the magnetic flux densities detected by the magnetic detection unit 43 and the magnetic detection unit 44 can include a disturbance component caused by geomagnetism, a magnetic field generated by a steel frame of a building, a magnetic field generated by the steel furniture, and the like, in addition to a magnetic field generated by magnetization. However, as described above, by obtaining the arithmetic mean of the magnetic flux densities detected by the two magnetic detection units, it is possible to reduce an influence of the disturbance component.

In a case in which a difference between the inner diameter of the through-hole 41 and the outer diameter of the insertion part 10 is made as small as possible, any one of the magnetic detection unit 43 or the magnetic detection unit 44 provided in the detection unit 40 is not essential and can be omitted. In this case, the processor 8P need only determine the movement direction, the movement distance, and the insertion length of the insertion part 10 on the basis of the magnetic flux densities BX and BY detected by the magnetic detection unit 43 or the magnetic detection unit 44.

In addition, in the present embodiment, each of the negative pole region 17S and the positive pole region 17N formed on the tubular member 17 is formed in an annular shape along the outer periphery of the tubular member 17. Therefore, even in a case in which the insertion part 10 is rotated in the circumferential direction thereof in the through-hole 41, it is possible to substantially eliminate the change in the magnetic flux densities detected by the magnetic detection unit 43 and the magnetic detection unit 44. Therefore, the movement direction, the movement distance, and the insertion length of the insertion part 10 can be determined regardless of the posture of the insertion part 10.

The disturbance component can be included in the magnetic flux densities detected by the magnetic detection unit 43 and the magnetic detection unit 44. In addition, the orientation of the disturbance component is also changed depending on the posture of the detection unit 40. Therefore, the influence of the disturbance component can be eliminated by classifying the magnetic flux density BX into two of the high level and the low level, classifying the magnetic flux density BY into three of the high level, the middle level, and the low level, and determining the movement state of the insertion part 10 in the longitudinal direction X on the basis of the combination of the classification levels as described above, rather than determining the movement state of the insertion part 10 in the longitudinal direction X using raw data of the magnetic flux density BX and the magnetic flux density BY as they are.

In the above description, the processor 8P classifies the magnetic flux density BX into two of the high level and the low level, classifies the magnetic flux density BY into three of the high level, the middle level, and the low level, and determines the movement state of the insertion part 10 in the longitudinal direction X on the basis of the combination of the classification levels. As a modification example, the processor 8P may classify the magnetic flux density BX into two of the high level and the low level, may classify the magnetic flux density BY into two of the high level and the low level, and may determine the movement state of the insertion part 10 in the longitudinal direction X on the basis of the combination of the classification levels.

Specifically, the processor 8P sets the “first threshold value th (for example, 0)” as the threshold value for classifying the magnetic flux density BX into two levels, and sets a “second threshold value th3 (for example, 0)” as the threshold value for classifying the magnetic flux density BY into two levels. Moreover, the processor 8P classifies the magnetic flux density BX by setting a value larger than the first threshold value th as the high level and setting a value less than the first threshold value th as the low level. Further, the processor 8P classifies the magnetic flux density BY by setting a value larger than the second threshold value th3 as the high level and setting a value less than the second threshold value th3 as the low level.

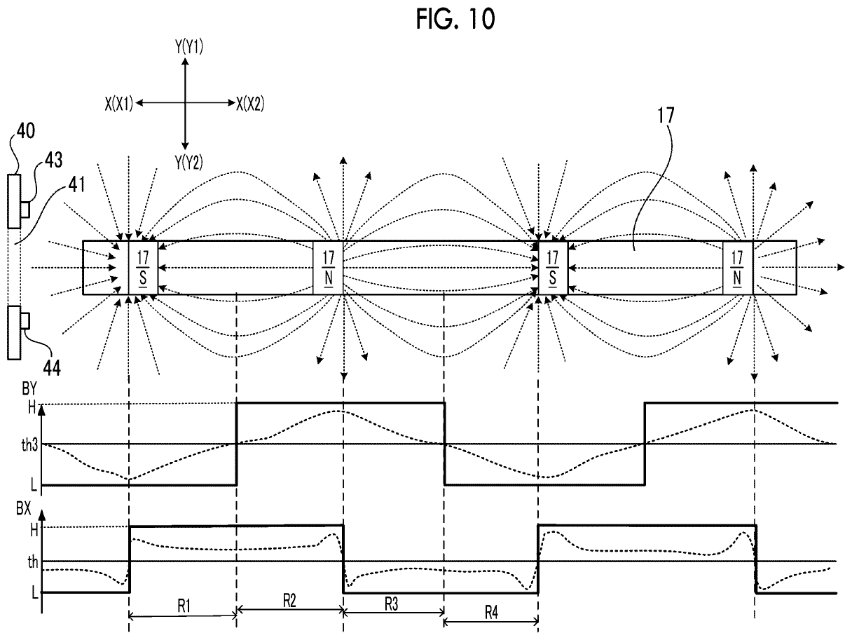

In FIG. 10, the result (classification level) of classifying the magnetic flux density BX and the magnetic flux density BY in the graphs illustrated in FIG. 8 is indicated by a thick solid line. As illustrated in FIG. 10, in the tubular member 17, a range between two adjacent positions P1 is divided into a region R1 in which the magnetic flux density BX is at the high level and the magnetic flux density BY is at the low level, a region R2 in which the magnetic flux density BX is at the high level and the magnetic flux density BY is at the high level, a region R3 in which the magnetic flux density BX is at the low level and the magnetic flux density BY is at the high level, and a region R4 in which the magnetic flux density BX is at the low level and the magnetic flux density BY is at the low level. As described above, the range between the negative pole ends adjacent to each other in the longitudinal direction X can be divided into four regions R1 to R4 depending on the combination of the classification level of the magnetic flux density BX and the classification level of the magnetic flux density BY. By monitoring the thick solid lines (classification levels of the magnetic flux densities BX and BY) illustrated in FIG. 10, the processor 8P can determine the movement direction of the insertion part 10 and the movement amount (movement distance) of the insertion part 10 in the longitudinal direction X.

In the description above, the processor 8P classifies the magnetic flux density into the plurality of pieces of information according to the magnitude thereof. However, a configuration may be adopted in which this classification is performed by a processor provided in the communication chip of the detection unit 40. That is, a configuration may be adopted in which the detection unit 40 transmits information on the classification level indicated by the thick solid line illustrated in FIG. 9 or FIG. 10 to the processor 8P. In addition, the processor 8P performs the determination of the movement state of the insertion part 10, but a configuration may be adopted in which the processor provided in the communication chip of the detection unit 40 performs the determination to transmit the determination result to the processor 8P. Further, a configuration may be adopted in which a processor such as a personal computer connected to the expansion device 8 via a network acquires the information on the magnetic flux density from the detection unit 40, performs the determination, and transmits the determination result to the processor 8P. Also, a processor separate from the processor 8P may perform the determination of the movement state of the insertion part 10. Further, a configuration may be adopted in which a processor provided outside the endoscope device 100 performs the determination of the movement state of the insertion part 10 to transmit the determination result to the processor 8P.

The threshold value used in a case of classifying each of the magnetic flux density BX and the magnetic flux density BY according to the magnitude thereof may be a predetermined fixed value, but the threshold value is preferably a variable value to be determined on the basis of the magnetic flux densities detected by the magnetic detection unit 43 and the magnetic detection unit 44 after the insertion of the insertion part 10 into the through-hole 41 is started.

For example, in a case in which the start-up switch of the detection unit 40 is turned on, the insertion part 10 is inserted into the through-hole 41, and the third magnetic pole region from the most distal end side of the tubular member 17 passes through the through-hole 41, the processor 8P can acquire each of the maximum value and the minimum value of the magnetic flux density BX detected by the magnetic detection unit 43, and the maximum value and the minimum value of the magnetic flux density BY detected by the magnetic detection unit 43. In a case where the maximum value and the minimum value of the magnetic flux density BX are acquired, the processor 8P obtains an average value of the maximum value and the minimum value, and sets the average value as the first threshold value th. Further, in a case in which the maximum value and the minimum value of the magnetic flux density BY are acquired, the processor 8P obtains an average value of the maximum value and the minimum value, sets a value obtained by adding a predetermined value to the average value as the second threshold value th1, and sets a value obtained by subtracting a predetermined value from the average value as the second threshold value th2. The predetermined value is a value that is larger than a value assumed as the disturbance component and is less than the absolute value of each of the maximum value and the minimum value of the magnetic flux density BY. The first to third magnetic pole regions from the most distal end side of the tubular member 17 constitute a base end part on the demagnetized region (adjacent region) side in the region in which the magnetic pattern is formed.

Hereinafter, the processor 8P need only classify the magnetic flux density BX and the magnetic flux density BY by using the threshold values set in this manner. In this way, it is possible to perform the determination of the movement state of the insertion part 10 with higher accuracy by setting the threshold values on the basis of the magnetic flux densities detected by the magnetic detection unit 43 and the magnetic detection unit 44.

In this way, in a case in which the threshold value is set on the basis of the magnetic flux densities detected by the magnetic detection unit 43 and the magnetic detection unit 44, it is preferable that, in a period until the third magnetic pole region from the most distal end side of the tubular member 17 passes through the through-hole 41, the processor 8P sets the first threshold value th, the second threshold value th1, and the second threshold value th2 to the predetermined values, performs the detection of the reference position and the determination of the movement state of the insertion part 10, and then updates the first threshold value th, the second threshold value th 1, and the second threshold value th2 by the method described above to perform the determination of the movement state of the insertion part 10.

As described above, in the endoscope system 200, the magnetic pattern is formed on the tubular member 17 such that the magnetic flux densities BX and BY detected by each of the magnetic detection unit 43 and the magnetic detection unit 44 are changed periodically between the positive side and the negative side and phases thereof are shifted in a case in which the insertion part 10 passes through the through-hole 41, so that it is possible to perform the determination of the movement state of the insertion part 10. Such a magnetic pattern is not limited to the configurations of the magnetic pole portions MA1 and MA2 illustrated in FIGS. 3 and 4, and can be variously modified.

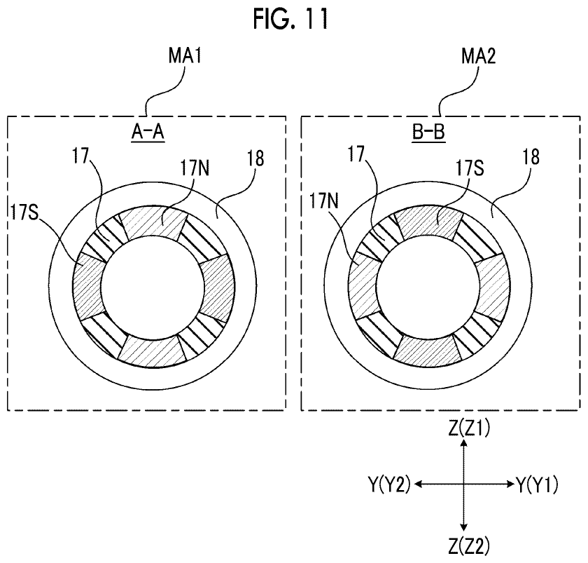

FIG. 11 is a schematic cross-sectional view illustrating a modification example of the magnetic pole portions MA1 and MA2 illustrated in FIG. 3 taken along the A-A arrow and the B-B arrow. In the modification example illustrated in FIG. 11, the magnetic pole portion MA1 has a configuration in which the negative pole region 17S and the positive pole region 17N are formed alternately with an interval therebetween along the circumferential direction of the tubular member 17. Similarly, the magnetic pole portion MA2 has a configuration in which the negative pole region 17S and the positive pole region 17N are formed alternately with an interval therebetween along the circumferential direction of the tubular member 17. The magnetic pole portion MA2 has a configuration in which the magnetic pole portion MA1 is rotated by 90 degrees around an axis center of the tubular member 17.

As illustrated in FIG. 11, in a state of being viewed in the longitudinal direction X, the positive pole region 17N in the magnetic pole portion MA1 and the negative pole region 17S in the magnetic pole portion MA2 are present at the same position in the circumferential direction of the tubular member 17. That is, in the tubular member 17, all the magnetic pole regions at the same position in the circumferential direction have a configuration in which the negative pole region 17S and the positive pole region 17N are alternately arranged in the longitudinal direction X. That is, in the tubular member 17, a first magnetic pattern in which the negative pole region 17S and the positive pole region 17N are alternately arranged along the longitudinal direction X with the negative pole region 17S at the beginning, and a second magnetic pattern in which the negative pole region 17S and the positive pole region 17N are alternately arranged along the longitudinal direction X with the positive pole region 17N at the beginning are alternately arranged with an interval therebetween in the circumferential direction of the tubular member 17.

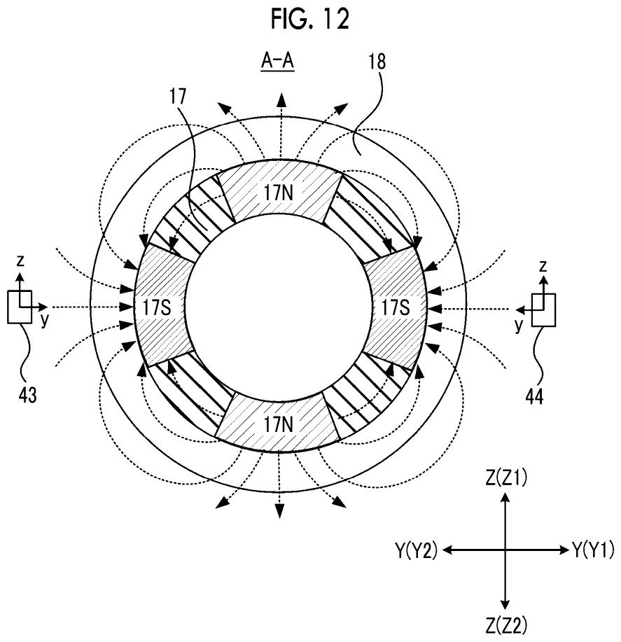

FIG. 12 is a diagram schematically illustrating a magnetic flux line generated in the magnetic pole portion MA1 having the configuration illustrated in FIG. 11. FIG. 12 illustrates the positions of the magnetic detection units 43 and 44 with respect to the soft portion 10A in a case in which the soft portion 10A passes through the through-hole 41.

In a state illustrated in FIG. 12, the magnetic flux density BY detected by the magnetic detection unit 43 has a large negative value. In a case in which the soft portion 10A is rotated by 45 degrees counterclockwise from the state illustrated in FIG. 12, the magnetic flux density BY detected by the magnetic detection unit 43 has a value close to zero. In a case in which the soft portion 10A is rotated by 90 degrees counterclockwise from the state illustrated in FIG. 12, the magnetic flux density BY detected by the magnetic detection unit 43 has a large positive value. In a case in which the soft portion 10A is rotated by 135 degrees counterclockwise from the state illustrated in FIG. 12, the magnetic flux density BY detected by the magnetic detection unit 43 has a value close to zero. In a case in which the soft portion 10A is rotated by 180 degrees counterclockwise from the state illustrated in FIG. 12, the magnetic flux density BY detected by the magnetic detection unit 43 has a large negative value. In a case in which the soft portion 10A is rotated in the circumferential direction thereof in the through-hole 41 in this manner, the magnetic flux density BY detected by the magnetic detection unit 43 is equivalent to the magnetic flux density BY illustrated in FIG. 8. Similarly, in a case in which the soft portion 10A is rotated in the circumferential direction thereof in the through-hole 41, the magnetic flux density BZ detected by the magnetic detection unit 43 is equivalent to the magnetic flux density BY illustrated in FIG. 8 and has a phase shifted by 90 degrees. Therefore, in a case in which the magnetic flux densities BY and BZ detected by the magnetic detection unit 43 are classified into the high level and the low level, respectively, these classification levels are equivalent to the thick solid lines of the magnetic flux density BY illustrated in FIG. 10 (it should be noted that the phases of the magnetic flux density BY and the magnetic flux density BZ are shifted by 90 degrees). Therefore, it is possible to derive a rotation direction and a rotation amount of the insertion part 10 by the combination of the classification levels.

In a case in which the endoscope 1 having the magnetic pattern having such a configuration is used, the processor 8P can determine a rotation state (rotation direction and rotation amount (rotation angle)) of the insertion part 10 in the circumferential direction in the same manner as the determination method of the movement state of the insertion part 10, by classifying each of the magnetic flux density BZ and the magnetic flux density BY into the plurality of pieces of information and monitoring the change in the combination of these pieces of information. In the configuration illustrated in FIG. 11, since the first magnetic pattern and the second magnetic pattern extending in the longitudinal direction X are formed on the tubular member 17, it is possible to determine the movement state of the insertion part 10 on the basis of the magnetic flux density BX and the magnetic flux density BY, as described above. In FIG. 11, each of the magnetic pole portions MA1 and the magnetic pole portions MA2 includes four magnetic pole regions arranged in the circumferential direction. However, each of the magnetic pole portion MA1 and the magnetic pole portion MA2 may have a configuration of including two magnetic pole regions, or have a configuration of including an even number (six or more) of magnetic pole regions.

Even in the configuration illustrated in FIG. 11, it is preferable that the arithmetic means of the magnetic flux densities BY and BZ detected by the magnetic detection unit 43 and the magnetic flux densities BY and BZ detected by the magnetic detection unit 44 are obtained, each of the values of these two arithmetic means is classified into the high level and the low level, and the rotation direction and the rotation amount of the insertion part 10 are derived by the combination of the classification levels.

### Processing of Processor 8P

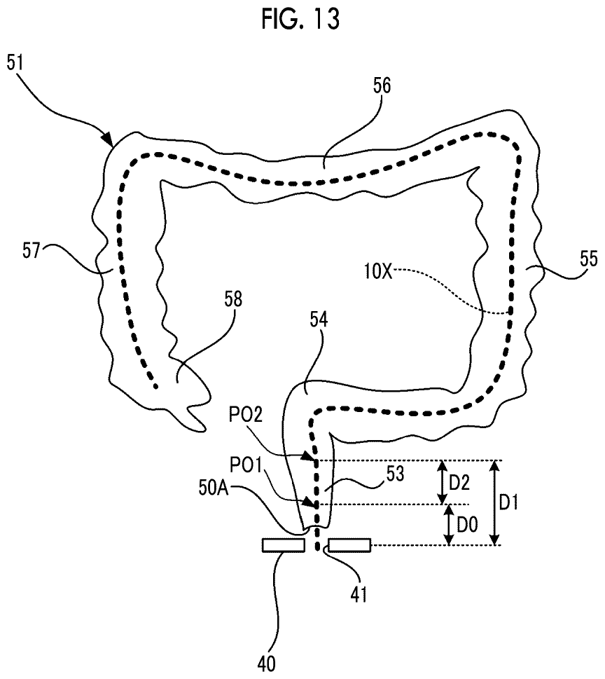

Next, details of various kinds of processing executed by the processor 8P will be described. In order to describe these various kinds of processing, the movement path of the insertion part 10 of the endoscope I will be described. FIG. 13 is a schematic diagram illustrating the movement path of the insertion part 10 in an examination (hereinafter, referred to as endoscopy) performed using the endoscope 1.

The endoscopy includes an endoscopy that examines an upper digestive organ such as a stomach, an endoscopy that examines a lower digestive organ such as a large intestine. In addition, the endoscopy includes a first examination in which the insertion part 10 is inserted into the subject in order to examine whether or not a lesion region is present in the subject, and a second examination in which the insertion part 10 is inserted into the subject in order to excise the already known lesion region.

### (Movement Path of Endoscope)

FIG. 13 illustrates a large intestine 51 of the subject (subject 50). In the endoscopy of the large intestine, the insertion part 10 is moved along a movement path 10X indicated by the broken line in the drawing. The movement path 10X is a tubular path from the through-hole 41 of the detection unit 40 disposed in the vicinity of the anus 50A outside the subject through the anus 50A to a rectum 53, and further from the rectum 53 through a sigmoid colon 54, a descending colon 55, a transverse colon 56, and an ascending colon 57 to an ileocecum 58.

In the endoscopy and the first examination of the large intestine, the operator of the endoscope 1 inserts the insertion part 10 into the anus 50A through the through-hole 41, causes the insertion part 10 to reach the ileocecum 58 which is a turning point of the examination, and then pulls out the insertion part 10 from the ileocecum 58 toward the outside of the subject. Hereinafter, a step of moving the distal end of the insertion part 10 from the through-hole 41 to the ilcocecum 58 will be described as an insertion step, and a step of moving the distal end of the insertion part 10 from the ileocecum 58 to the through-hole 41 will be referred to as a pulling-out step. The first examination is composed of a set of the insertion step and the pulling-out step. The endoscopy and the second examination of the large intestine are the same as the first examination except that the turning point of the examination is changed to the presence position of the lesion region found in the first examination in advance.

In the endoscopy of the stomach, the turning point of the first examination is the duodenum, and the turning point of the second examination is the presence position of the lesion region found in the first examination in advance.

### (Processing During Endoscopy)

In a case where the endoscopy is started, the power of the detection unit 40 is turned on. As described above, the processor 8P derives a first distance (the insertion length described above) from the reference position (position of the through-hole 41) on the movement path 10X to the distal end of the insertion part 10 on the basis of the magnetic flux densities BX and BY detected by the detection unit 40.

In a case in which the endoscope 1 is activated, the processor 8P performs reaching site recognition processing of sequentially acquiring the captured images captured by the endoscope 1, and recognizing the site (the anus, the rectum, the sigmoid colon, the top part of the sigmoid colon (S-top), the transition part between the sigmoid colon and the descending colon (SDJ), the descending colon, the splenic flexure, the transverse colon, the hepatic curvature, the ascending colon, the ileocecum, or the outside of the body and the like) in the subject that the distal end of the insertion part 10 has reached on the basis of the acquired captured images. The processor 8P performs the reaching site recognition processing using, for example, a recognition model generated by machine learning that outputs a recognition result of the site in the subject with the captured image as an input. Alternatively, the processor 8P may recognize to which site in the subject the distal end of the insertion part 10 has reached, on the basis of the information input from the operator of the endoscope 1. For example, in a case where the operator recognizes from the captured image that the distal end of the insertion part 10 has reached a predetermined site, the operator performs, for example, a predetermined operation on the endoscope 1 to input information indicating that the predetermined site has been reached. Upon receiving the information, the processor 8P recognizes that the site in the subject that the distal end of the insertion part 10 has reached is the predetermined site.

The processor 8P can also determine, for example, whether any of the insertion step or the pulling-out step is performed by using the result of the reaching site recognition processing. As an example, the processor 8P determines a period after the recognition result that the reaching site is the anus 50A or the rectum 53 is obtained until the recognition result that the reaching site is the ileocecum 58 is obtained, as a period (first period) of the insertion step in which the endoscope 1 is moved from a starting point toward an ending point of the movement path 10X, and determines a period after the recognition result that the reaching site is the ilcocecum 58 is obtained until the recognition result that the reaching site is the outside of the subject is obtained, as a period (second period) of the pulling-out step in which the endoscope 1 is moved from the ending point toward the starting point of the movement path 10X.

The processor 8P can determine the movement direction of the insertion part 10 on the movement path 10X on the basis of a time change of the first distance derived on the basis of the magnetic flux densities BX and BY detected by the detection unit 40, and can discriminate the period of the insertion step and the period of the pulling-out step from the movement direction. For example, in a case where the first distance tends to be increased, the processor 8P determines that the insertion part 10 is being moved in a direction from the outside of the body of the subject toward the ileocecum 58, and determines the period of the insertion step (first period). On the other hand, in a case where the first distance tends to be decreased, the processor 8P determines that the insertion part 10 is being moved from the ileocecum 58 toward the outside of the body of the subject, and determines the period of the pulling-out step (second period).

### (Detection of Event)

The processor 8P can detect the occurrence of various events related to the endoscopy by using, for example, the result of the above-described reaching site recognition processing and the result of the above-described lesion recognition processing and treatment tool recognition processing to acquire event information which is information on the event.

For example, the processor 8P can detect an event that the insertion step is started, an event that the pulling-out step is started, an event that the endoscopy is ended, an event that the distal end of the endoscope I reaches a specific site in the subject, an event that a specific operation (for example, operation of the treatment tool) of the endoscope 1 is performed, an event that the lesion region is detected from the subject, or the like.

Specifically, in a case where the recognition result that the reaching site is the anus 50A is obtained by the reaching site recognition processing, the processor 8P detects the occurrence of the event that the endoscopy is started (insertion step is started) (examination start event). In a case where, after the examination start event is detected, the recognition result that the reaching site is the ileocecum 58 is obtained by the reaching site recognition processing, the processor 8P detects the occurrence of the event that the pulling-out step is started (pulling-out start event). In a case where, after the pulling-out start event, the recognition result that the reaching site is not inside the subject is obtained, the processor 8P detects the occurrence of the event that the endoscopy is ended (examination end event).

In a case where the lesion region is detected by the lesion recognition result, the processor 8P detects the occurrence of the event that the lesion region is detected (lesion detection event). In a case where the treatment tool is detected by the treatment tool recognition result, the processor 8P detects the occurrence of the event that the treatment (operation of the treatment tool) is performed (treatment event). In a case where the recognition result that a predetermined specific site is reached is obtained by the reaching site recognition processing, the processor 8P detects the event that the distal end of the insertion part 10 reaches the specific site (specific site reaching event).

The processor 8P can detect an event that a specific operation (for example, the hardness adjustment of the insertion part 10) of the endoscope 1 is performed on the basis of the information input from the operator, to acquire information on the event.

The processor 8P can also detect the occurrence of an operator operation event that the operator performs a specific operation to acquire information on the event. For example, in a case where the operator performs, as a specific operation, rotation (twisting) of the insertion part 10, manual compression, or the like, the operator performs a voice input, an operation of an input device such as a touch panel, or a button operation of the endoscope 1 to input information indicating that the operation is performed. By receiving this information, the processor 8P can detect the occurrence of an event that the operation is performed. By adopting the magnetic pattern illustrated in FIG. 11, the processor 8P can also detect that the rotation of the insertion part 10 is performed on the basis of the information on the magnetic flux density detected by the detection unit 40.

In addition, the processor 8P can detect the examination start event, the pulling-out start event, the examination end event, the lesion detection event, the treatment event, and the specific site reaching event on the basis of the information input from the operator without using the results of the reaching site recognition processing, the lesion recognition processing, and the treatment tool recognition processing. For example, in a case of the occurrence of various events such as the examination start (insertion start), the pulling-out start, the examination end (pulling-out end), the lesion region detection, the treatment execution, reaching the specific site, and the like, the operator performs the voice input, the operation of the input device such as a touch panel, the button operation of the endoscope 1, and the like. By these operations, the processor 8P can detect the occurrence of the event to acquire the event information.

### (Derivation of Second Distance)

The processor 8P derives a second distance as a distance between the distal end of the insertion part 10 and the specific site in the subject on the basis of the result of the above-described reaching site recognition processing and the first distance derived on the basis of the magnetic flux densities BX and BY.

First, in a case where the endoscopy of the large intestine is started, in the initial stage of the insertion step, the processor 8P obtains the recognition result that the reaching site of the distal end of the insertion part 10 is the anus 50A or the rectum 53. In a case where such a recognition result is obtained, the processor 8P sets the first distance derived in a state where the recognition result is obtained, as a first correction value. Then, after the recognition result is obtained, the processor 8P performs processing of obtaining a specific insertion length (insertion length of the insertion part 10 in a case where the anus 50A or the rectum 53 on the starting point side of the movement path 10X is the reference position) by subtracting the first correction value from the first distance derived on the basis of the magnetic flux densities BX and BY. By this processing, in the insertion step, the second distance in a case where the anus 50A or the rectum 53 is the specific site (first specific site) is sequentially derived as the specific insertion length. The specific insertion length constitutes a first value. For example, as illustrated in FIG. 13, a case is assumed in which the recognition result that the reaching site is the rectum 53 in a state where the distal end of the insertion part 10 reaches a position PO1 is obtained. In this case, in a case where the distal end of the insertion part 10 slightly advances from the rectum 53 to be moved to a position PO2, a value (=D2) obtained by subtracting the first distance (=D0, first correction value) derived in a state where the distal end of the insertion part 10 is at the position PO1, from the first distance (=D1) derived at a point in time when the distal end of the insertion part 10 is moved to the position PO2, is derived as the specific insertion length.

After that, in a case where the insertion step is continued and the distal end of the insertion part 10 is moved to a turning point (that is, the ileocecum 58) at which the insertion step is switched to the pulling-out step, the processor 8P obtains the recognition result that the reaching site of the distal end of the insertion part 10 is the ileocecum 58. In a case where the recognition result that the reaching site is the ilcocecum 58 is obtained, the processor 8P sets the first distance derived in the state where the recognition result is obtained, as a second correction value. Then, after the recognition result is obtained, the processor 8P performs processing of obtaining the pulling-out length (pulling-out length of the insertion part 10 in a case where the ileocecum 58 as the ending point of the movement path 10X is the reference position) by subtracting the first distance derived on the basis of the magnetic flux densities BX and BY from the second correction value. By this processing, in the pulling-out step, the second distance in a case where the ileocecum 58 is the specific site (second specific site) is sequentially derived as the pulling-out length. The pulling-out length constitutes a second value.

In the insertion step of the endoscopy of the large intestine, the insertion part 10 may be inserted while the large intestine is folded, or the insertion part 10 may be inserted while the large intestine is stretched. On the other hand, in the pulling-out step of the endoscopy of the large intestine, the insertion part 10 is pulled out in a state where the large intestine has returned to a steady state. Therefore, in the endoscopy of the large intestine, even in a case where the first distances derived on the basis of the magnetic flux densities BX and BY are the same in the insertion step and the pulling-out step, the positions at which the distal end of the insertion part 10 is present in the large intestine 51 are different in some cases. In the present embodiment, in the insertion step, the front end position of the insertion part 10 can be managed by the specific insertion length, and in the pulling-out step, the front end position of the insertion part 10 can be managed by the pulling-out length. Therefore, the insertion state of the insertion part 10 can be managed with high accuracy.

The specific insertion length constitutes a distance from the reference position (position of the anus 50A or the rectum 53) on the starting point side of the movement path 10X to the distal end of the endoscope 1 moved along the movement path 10X. The pulling-out length constitutes a distance from the ending point position (the position of the ileocecum 58) on the movement path 10X to the distal end of the endoscope 1 moved along the movement path 10X. The first distance constitutes a distance from the reference position (position of the through-hole 41) on the starting point side of the movement path 10X to the distal end of the endoscope 1 moved along the movement path 10X. The first distance, the specific insertion length, or the pulling-out length will also be referred to distance information below.

In addition, the processor 8P may derive the specific insertion length, which is an insertion length of the insertion part 10 with the anus or the rectum as a reference position, even in the pulling-out step. That is, the processor 8P can perform any of processing of deriving the first distance and the specific insertion length in the insertion step and the first distance and the pulling-out length in the pulling-out step, or processing of deriving the first distance and the specific insertion length in each of the insertion step and the pulling-out step.

### (Display and Recording During Endoscopy)

In the period of the insertion step, it is preferable that the processor 8P performs control to display at least one of the specific insertion length (second distance) or the first distance derived as described above on the display device 7, or performs control to record the specific insertion length or the first distance in association with the information regarding the endoscopy (hereinafter, referred to as examination association information) in the recording medium (for example, the memory of the expansion device 8). The examination association information refers to the captured image captured by the endoscope 1, various kinds of event information described above, an elapsed time (examination time) from the start of the endoscopy (examination start event), and the like. For example, each time the first distance and the specific insertion length are derived, the processor 8P performs control to record the derived value in association with the elapsed time (examination time). In a case where there is an instruction to record the captured image, the processor 8P performs control to record the captured image further in association with the elapsed time at that time. In a case where the event information is acquired, the processor 8P performs control to record the event information in association with the elapsed time at that time.

In the period of the pulling-out step, it is preferable that the processor 8P performs control to display at least one of the pulling-out length (the second distance) or the first distance derived as described above on the display device 7, or performs control to record the pulling-out length or the first distance in association with the examination association information in the recording medium.

In any of the insertion step or the pulling-out step, the processor 8P may perform control to display only the first distance among the first distance, the specific insertion length, and the pulling-out length on the display device 7, and may further use the first distance, the specific insertion length, and the pulling-out length for other uses other than the display. Specifically, the processor 8P may perform control to record at least one of the first distance, the specific insertion length, or the pulling-out length in association with the examination association information in the recording medium, or perform control to output operation support information of the endoscope 1 on the basis of at least one of the first distance, the specific insertion length, or the pulling-out length.

For example, in the insertion step, depending on the position of the distal end of the insertion part 10, the hardness adjustment of the insertion part 10 of the endoscope 1 or the manual compression may be required in order to smoothly insert the insertion part 10. For example, in a case where it is determined that the distal end of the insertion part 10 reaches a position where the hardness adjustment or manual compression is required, from the derived specific insertion length, the processor 8P performs control to display the information (operation support information) instructing the hardness adjustment or manual compression on the display device 7, or performs control to output the information (operation support information) instructing the hardness adjustment or manual compression by voice from a speaker. In this manner, it is possible to smoothly insert the endoscope 1. Among the insertion step and the pulling-out step, the processor 8P may perform control to output the operation support information on the basis of the first distance or the specific insertion length only in the insertion step, and may not perform the control in the pulling-out step. In the pulling-out step of the endoscopy of the large intestine, it is often not difficult to pull out the endoscope 1, and therefore, it is possible to reduce the processing load of the processor 8P by doing in this manner.

The processor 8P can determine the position (site) that the distal end of the insertion part 10 has reached by using the derived first distance, specific insertion length, or pulling-out length and table data recorded in advance. For example, table data indicating a correspondence relationship between the first distance and the front end position of the endoscope 1 in the large intestine, table data indicating a correspondence relationship between the specific insertion length and the front end position of the endoscope 1 in the large intestine, and table data indicating a correspondence relationship between the pulling-out length and the front end position of the endoscope 1 in the large intestine can be statistically obtained from actual data of a large number of times of endoscopy performed on various subjects, or can be statistically obtained from actual data of the endoscopy performed on the same subject, and can be recorded in the recording medium (for example, the memory of the expansion device 8) accessible by the processor 8P. The processor 8P can determine the position that the distal end of the insertion part 10 has reached, by acquiring the information on the front end position (reaching site) of the endoscope 1 corresponding to the first distance, the specific insertion length, or the pulling-out length derived at the time of the endoscopy, from the table data.

The examination data including the examination association information (the captured image, the event information, or the examination time) and the distance information (the first distance, the specific insertion length, or the pulling-out length) associated by the processor 8P is transmitted to a server (not illustrated) and stored. After the endoscopy is ended, an examination report creation support device that can access the server creates a draft of an examination report on the basis of the examination data. A doctor can efficiently perform work by creating a final examination report using the draft.

### (Display Example of Examination Data)

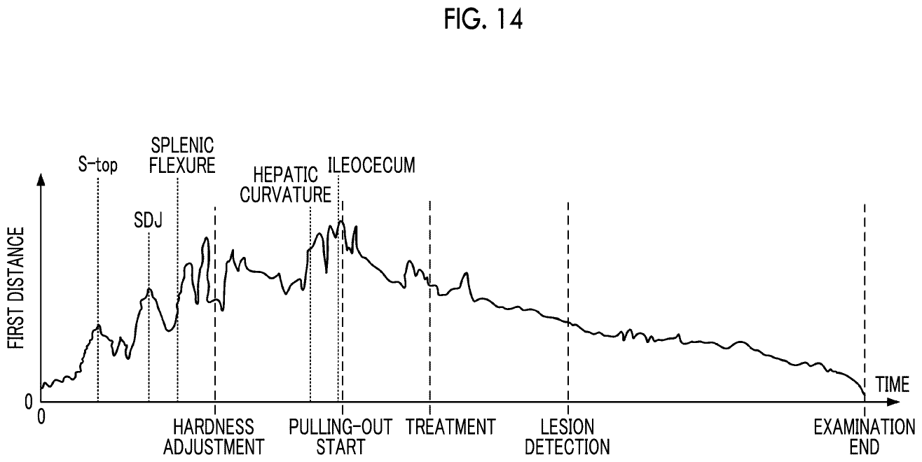

FIG. 14 is a graph illustrating a display example of the examination data associated and recorded by the processor 8P. The processor 8P performs control to display the graph illustrated in FIG. 14 on, for example, the display device 7 or another display. With the graph displayed in this way, the operator of the endoscope 1 or an instructor thereof can evaluate the procedure of the endoscopy.

In the graph illustrated in FIG. 14, the first distance is plotted for each elapsed time of the endoscopy. In the graph illustrated in FIG. 14, characters (S-top, SDJ, splenic flexure, hepatic curvature, and ileocecum) indicating the content (reaching site) of the specific site reaching event are attached to the timing when the specific site reaching event is detected. In addition, characters (hardness adjustment, pulling-out start, treatment, lesion detection, examination end) indicating the content of another event are attached to the timing at which the event is detected. The period from the start of the insertion step (elapsed time=0) to the pulling-out start event is the period of the insertion step, and the period from the pulling-out start event to the examination end event is the period of the pulling-out step.

In the graph illustrated in FIG. 14, the vertical axis may indicate the distance, the specific insertion length may be plotted in the period of the insertion step, and the pulling-out length may be plotted in the period of the pulling-out step. Alternatively, in a case where a specific insertion length is derived instead of the pulling-out length in the pulling-out step, the vertical axis may indicate the specific insertion length, and the specific insertion length may be plotted in each period of the insertion step and the pulling-out step.

In a case where an arbitrary position in the plot waveform of the distance information in FIG. 14 is designated and a captured image associated with the elapsed time of the arbitrary position is recorded, the processor 8P may display the captured image on the display device 7. The processor 8P may perform control to display the graph illustrated in FIG. 14 and a reference graph (graph in which the distance information is plotted for each elapsed time and an occurrence timing of the specific site reaching event is added) based on reference data (data in which the elapsed time, the distance information, and the event information are associated with each other) generated in advance, in a comparable manner on the display device 7.

The reference data can be data that is statistically generated on the basis of the examination data obtained by each of the plurality of times of endoscopy, past examination data obtained by the endoscopy performed by an operator who has a high evaluation in procedure, examination data of the past endoscopy performed by the same operator as that in the endoscopy of the graph illustrated in FIG. 14, or the like.

The processor 8P may perform control to display the graph illustrated in FIG. 14 on the display device 7 in real time during the endoscopy. In this case, the processor 8P acquires the reference data recorded in the memory or the like of the server before the start of the endoscopy, and displays the reference graph based on the acquired reference data on the display device 7. In a case where the endoscopy is started after the reference graph is displayed on the display device 7, it is preferable that the processor 8P detects a timing at which the distance information at the time of “elapsed time =0” in the displayed reference graph and the distance information derived after the start of the endoscopy match each other, as an examination start timing, starts to plot the distance information, and displays a graph corresponding to the endoscopy being performed.

In a case where an operation of requesting correction of at least one of the elapsed time or the event information is performed for the graph illustrated in FIG. 14, it is preferable that the processor 8P receives the correction, and corrects at least one of the elapsed time or the event information in the examination data corresponding to the graph. Thereby, for example, the “hardness adjustment” illustrated in FIG. 14 can be changed to “manual compression”, the timing of the “hardness adjustment” illustrated in FIG. 14 can be shifted to the left or right, or both of them can be performed. In this manner, it is possible to more accurately leave the correspondence relationship among the elapsed time, the distance information, and the event information.

It is preferable that the processor 8P generates table data (correspondence table of the reaching site and the distance information) indicating the statistical correspondence between the event information (information on the specific site reaching event) and the distance information on the basis of the examination data acquired by each of the plurality of times of endoscopy, and records the table data in the memory or the like of the expansion device 8. Specifically, processing of extracting the distance information in a case where the distal end of the endoscope 1 has reached the specific site in the large intestine from the examination data of the insertion step obtained in each time of endoscopy, calculating a representative value (for example, average value or median value) of all pieces of the extracted distance information, and associating the specific site with the representative value thereof is repeatedly performed while changing the specific site, and thereby the first table data indicating the correspondence between the distance information and each specific site in the large intestine is generated.

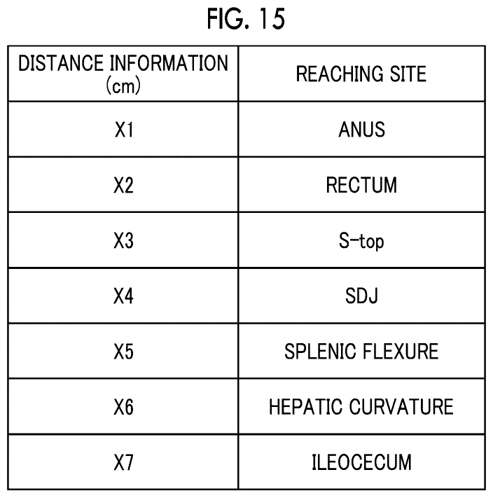

FIG. 15 is a diagram illustrating an example of the first table data. The distance information in the first table data illustrated in FIG. 15 is the first distance or the specific insertion length. The first table data may be generated separately for the insertion step and the pulling-out step. In this case, the distance information of the first table data for the insertion step is the specific insertion length, and the distance information of the first table data for the pulling-out step is the pulling-out length. Data indicating the correspondence relationship between the distance information and the site in the subject can also be generated according to the anatomical information without using the examination data.

The processor 8P can determine the position of the distal end of the endoscope 1 in the large intestine by using the distance information derived during the endoscopy and the first table data exemplified in FIG. 15.

### Main Effects of Endoscope System 200

With the endoscope system 200, not only the insertion length (first distance) of the insertion part 10 into the subject with the position of the detection unit 40 installed outside the subject as the starting point, but also the specific insertion length of the insertion part 10 into the subject with the first specific site (anus or rectum) in the subject as the starting point and the pulling-out length of the insertion part 10 to the outside of the subject with the second specific site (ilcocecum) in the subject as the starting point can be derived. In a case of performing the endoscopy of the stomach, it is possible to obtain the specific insertion length and the pulling-out length by setting the first specific site as, for example, a cardia, and the second specific site as, for example, a duodenum.

With the endoscope system 200, since the specific insertion length and the pulling-out length are derived by using the result of the reaching site recognition processing using the captured image obtained through the imaging by the endoscope 1 actually inserted into the subject, it is possible to eliminate the influence of individual differences for each subject, and manage the front end position of the insertion part 10 with high accuracy by using the specific insertion length and the pulling-out length. As a result, it is possible to perform the operation support of the endoscope 1 with high accuracy during the endoscopy. In addition, it is possible to determine the recording position of the captured image with high accuracy, which can be used for later creation of an examination report or can improve the diagnosis accuracy. In particular, since the specific insertion length and the pulling-out length can be derived separately, these effects can be further enhanced.

### Preferred Embodiment I: Insertion Control by Specific Insertion Length

In a case where the insertion part 10 of the endoscope 1 has a self-propelled mechanism, the processor 8P may perform drive control of the mechanism on the basis of the derived specific insertion length or first distance in the insertion step. For example, the processor 8P drives the mechanism such that the time change of the specific insertion length or the first distance derived in the insertion step is equal to the time change (for example, time change of the specific insertion length or the first distance in the reference data described above) of the statistically calculated specific insertion length or first distance at the time of the ideal insertion of the endoscope, to perform control to move the insertion part 10 along the movement path 10X. In this manner, it is possible to efficiently insert the endoscope 1 into the subject regardless of the skill level of the operator.

### Preferred Embodiment II: Processing Based on Change in Position of Detection Unit

Since the detection unit 40 is disposed outside the subject as illustrated in FIG. 1, the position (the reference position used for derivation of the first distance) of the detection unit 40 in a direction along the movement path 10X can be changed during the endoscopy. For example, in a case where the position of the detection unit 40 is moved in a direction further away from the subject than at the start of the insertion step, the derived first distance is increased by the movement distance of the detection unit 40, and as a result, the specific insertion length or the pulling-out length has an error corresponding to the movement distance. On the other hand, in a case where the position of the detection unit 40 is moved in a direction closer to the subject than at the start of the insertion step, the derived first distance is decreased by the movement distance of the detection unit 40, and as a result, the specific insertion length or the pulling-out length has an error corresponding to the movement distance. Therefore, it is preferable to determine the presence or absence of such a change in position of the detection unit 40, and in a case where there is a change in position, to correct an error of the specific insertion length or the pulling-out length due to the change in position.

For example, the processor 8P acquires a change amount per unit time of the first distance derived during the endoscopy (difference between the first distance (referred to as distance LL1) derived at timing t1 and the first distance (referred to as distance LL2) derived at timing t2 after timing t1). In addition, the processor 8P acquires the movement amount of the captured image (the movement amount between the captured image captured at timing t1 and the captured image captured at timing t2) in the period in which the change amount is acquired. The movement amount of the captured image is a change amount in the brightness of the captured image, a movement amount of a feature point extracted from the captured image, or the like. The processor 8P determines the presence or absence of the change in position of the detection unit 40 on the basis of the change amount and the movement amount.

For example, the processor 8P compares a conversion value obtained by converting the movement amount of the captured image into a distance in a direction along the movement path 10X with the change amount of the first distance, determines that there is a change in position of the detection unit 40 in a case where the difference between the conversion value and the change amount is equal to or greater than a threshold value, and determines that there is no change in position of the detection unit 40 in a case where the difference between the conversion value and the change amount is less than the threshold value. In a case where it is determined that there is a change in position of the detection unit 40, the processor 8P corrects the specific insertion length or the pulling-out length so as to offset the amount of the change in position of the detection unit 40. By such processing, it is possible to derive the specific insertion length or the pulling-out length with high accuracy even in a case where there is a change in position of the detection unit 40. In a case where it is determined that there is a change in position of the detection unit 40, the processor 8P may output warning information from the display device 7, the speaker, or the like. In this manner, it is possible to prompt the operator to adjust the position of the detection unit 40.

### Preferred Embodiment III: Notification by Target Position

It is preferable that the processor 8P performs control of giving a notification on the basis of at least one of the first distance, the specific insertion length, or the pulling-out length derived during the endoscopy, and target position information indicating the target position of the movement path 10X, which is recorded in advance in the memory or the like of the expansion device 8. The control of giving a notification means displaying predetermined information on the display device 7, or outputting predetermined information from a speaker (not illustrated). By this control, the predetermined information is provided to a person involved in the endoscopy.

For example, the target position information can be the information on the first distance or the information on the specific insertion length and the pulling-out length acquired by the processor 8P in a state where the endoscope 1 captures the region-of-interest in the subject in the past endoscopy (for example, the preliminary first examination in a case of performing the second examination) performed on the same subject.

For example, in the insertion step of the first examination in the endoscopy of the large intestine for a certain subject (referred to as subject H), a case is assumed in which, in a case where the operator checks a captured image and there is a region suspected to be a lesion in the captured image, an operation of recording the region as the region-of-interest is performed. In this case, in a case where the operation of recording the region-of-interest is performed by the operator, the processor 8P records the first distance or the specific insertion length derived at the time at which the operation is performed or derived most recently at that time, as the target position information in the memory of the expansion device 8. In addition, in the pulling-out step of the first examination in the endoscopy of the large intestine for the subject H, a case is assumed in which, in a case where the operator checks a captured image and there is a region suspected to be a lesion in the captured image, an operation of recording the region as the region-of-interest is performed. In this case, in a case where the operation of recording the region-of-interest is performed by the operator, the processor 8P records the first distance or the pulling-out length derived at the time at which the operation is performed or derived most recently at that time, as the target position information in the memory of the expansion device 8.

In a case where the first examination for the subject H is ended and then the second examination for the subject H is started, the processor 8P performs control of giving a notification of the presence of the region-of-interest in a case where the first distance, the specific insertion length, or the pulling-out length sequentially derived becomes a value close to the above-described target position information acquired from the memory of the expansion device 8. For example, in the insertion step of the second examination, the processor 8P performs control of giving a notification of the presence of the region-of-interest in a case where the derived first distance or specific insertion length substantially matches the target position information (the first distance or the specific insertion length) recorded in the memory of the expansion device 8. In addition, in the pulling-out step of the second examination, the processor 8P performs control of giving a notification of the presence of the region-of-interest in a case where the derived first distance or pulling-out length substantially matches the target position information (the first distance or the pulling-out length) recorded in the memory of the expansion device 8.

In a case where the specific insertion length is derived also in the pulling-out step, in the same endoscopy, the processor 8P may perform recording of the target position information in the insertion step, and perform notification on the basis of the target position information in the pulling-out step. For example, in the insertion step of the endoscopy, in a case where the operator performs an operation of recording the region-of-interest, the processor 8P records the specific insertion length derived at the time at which the operation is performed or derived most recently at that time, as the target position information in the memory of the expansion device 8. After that, in a case where the pulling-out step in the same endoscopy is started, the processor 8P performs control of giving a notification of the presence of the region-of-interest on the basis of the specific insertion length derived in the pulling-out step and the target position information (specific insertion length) recorded in the memory of the expansion device 8 in the insertion step.

Specifically, the processor 8P sets a predetermined range of the specific insertion length including the target position information recorded in the insertion step, on the movement path 10X, and notifies, in the pulling-out step, of the presence of the region-of-interest in a case where the derived specific insertion length enters the predetermined range. For example, it is assumed that the target position information recorded in the insertion step is the specific insertion length and the value thereof is a distance XX1. In this case, a range between a value obtained by adding an arbitrary value ΔXX1 to the distance XX1 and a value obtained by subtracting the value ΔXX1 from the distance XX1 is set as the predetermined range. Then, in the pulling-out step, in a case where the derived specific insertion length enters the predetermined range, the control of giving a notification of the presence of the region-of-interest is performed. By doing so, it is possible to perform the notification with high accuracy in consideration of the difference in the position of the distal end of the endoscope 1 in the large intestine between the insertion step and the pulling-out step.

As another case, in the insertion step in the endoscopy of the large intestine for the subject H, a case is assumed in which, in a case where the operator checks a captured image and there is a region suspected to be a lesion in the captured image, an operation of recording the region as the region-of-interest is performed, and then, in a case where the insertion of the endoscope 1 proceeds and there is a region as a marker, an operation of recording the region as the region-of-interest is performed.

In this case, in a case where the operation of recording the lesion region is performed by the operator, the processor 8P records distance information La (the first distance or the specific insertion length) derived at the time at which the operation is performed or derived most recently at that time, in the memory of the expansion device 8. Then, in a case where the operator performs an operation of recording the region as a marker, the processor 8P calculates the difference Al (absolute value) between distance information Lb (the first distance or the specific insertion length) derived at the time at which the operation is performed or derived most recently at that time and the distance information La recorded in advance, and records a distance Lc obtained by subtracting a value slightly smaller than the difference Δ1 from the distance information Lb, as the target position information in the memory. In addition, the processor 8P records a captured image IM1 in a case where the operation is performed, in the memory. After that, in a case where the pulling-out step is started and a captured image similar to the captured image IM1 is acquired, the processor 8P sets the first distance or the specific insertion length at that time as the reference value, and in a case where the first distance or the specific insertion length derived thereafter becomes a value smaller than the reference value by the distance Lc, the processor 8P performs control of giving a notification of the presence of the region-of-interest.

As described above, by giving a notification of the presence of the region-of-interest on the basis of the target position information, and the first distance or the specific insertion length and the pulling-out length, it is possible to improve the examination accuracy and improve the examination efficiency by preventing the operator from overlooking the lesion region.

### Preferred Embodiment IV: Notification by Target Position and Image Recognition Result

It is preferable that the processor 8P performs control of giving a notification on the basis of the first distance or the specific insertion length and the pulling-out length derived during the endoscopy, the above-described target position information recorded in the memory or the like of the expansion device 8, and the result of the lesion recognition processing.

For example, in a state where the first distance, the specific insertion length, or the pulling-out length derived in the endoscopy substantially matches the target position information (the first distance, the specific insertion length, or the pulling-out length) recorded in the memory of the expansion device 8, the processor 8P acquires a captured image captured in that state or in the vicinity of a timing of reaching that state by the endoscope 1, and performs the lesion recognition processing on the basis of the captured image. Further, as a result of the lesion recognition processing, the processor 8P performs control of giving a notification of the presence of the region-of-interest in a case where the lesion region is detected, and the processor 8P does not perform the control in a case where the lesion region is not detected. In this manner, by further using the result of the lesion recognition processing, it is possible to determine with high accuracy whether or not the distal end of the endoscope 1 has reached the presence position of the lesion region, which is the target position, and thereby more accurate notification can be made.

### Modification Example of Target Position Information

The target position information can be distance information that can be acquired by the processor 8P in a state where the distal end of the endoscope 1 is located on the starting point side of the movement path 10X. In this case, the processor 8P performs control of giving a notification of the information regarding the end of the endoscopy on the basis of the distance information derived in the endoscopy, and the target position information acquired from the memory. The information regarding the end of the endoscopy is information indicating that the endoscopy is nearing the end, information prompting the start of work (calling to the subject using sedatives, preparatory work for cleaning the endoscope, preparatory work for the next endoscopy, and the like) following the end of the endoscopy, or the like.

Such target position information is generated by the processor 8P statistically processing (for example, averaging a large number of pieces of the distance information) the information on the first distance, the pulling-out length, or the specific insertion length (limited to a case where the specific insertion length is derived instead of the pulling-out length in the pulling-out step) derived at a timing before the predetermined time from the detection timing of the examination end event, in each of the plurality of times of endoscopy performed in the past by the same operator or a plurality of operators, for example.

For example, in the pulling-out step, in a case where the difference between the pulling-out length and the target position information (pulling-out length) is equal to or less than the threshold value, or in a case where the pulling-out length is greater than the target position information (pulling-out length), the processor 8P determines that the distal end of the endoscope 1 has reached the position in the vicinity of the outside of the subject, and performs control of giving a notification of the information regarding the end of the endoscopy. Alternatively, in the pulling-out step, in a case where the difference between the first distance or the specific insertion length and the target position information (the first distance or the specific insertion length) is equal to or less than the threshold value, the processor 8P determines that the distal end of the endoscope 1 has reached the position in the vicinity of the outside of the subject, and performs control of giving a notification of the information regarding the end of the endoscopy. Accordingly, a person concerned who has checked the information can recognize that the end of the endoscopy is approaching, and start various works, so that the endoscopy can be performed efficiently.

It is preferable that in a case where the difference between the pulling-out length and the target position information (pulling-out length) is equal to or less than the threshold value, or in a case where the difference between the first distance or the specific insertion length and the target position information (the first distance or the specific insertion length) is equal to or less than the threshold value, the processor 8P determines the movement direction of the insertion part 10, in a case where it is determined that the determined movement direction is a direction from the inside toward the outside of the subject, the processor 8P performs control of giving a notification of the information regarding the end of the endoscopy, and in a case where it is determined that the determined movement direction is a direction from the outside toward the inside of the subject, the processor 8P does not perform the control. In this manner, it is possible to prevent the information regarding the end of the endoscopy from being notified in the insertion step.

In a case where the occurrence of the lesion detection event is detected after the control of giving a notification of the information regarding the end of the endoscopy is performed, it is preferable that the processor 8P changes the notification content. The change of the notification content includes stopping the notification, correction of the remaining time in a case where the remaining time until the end of the examination is notified, and the like. In this manner, in a case where a region to be noticed such as the lesion region is detected after the control of giving a notification of the information regarding the end of the endoscopy is performed, it is possible to prompt the persons concerned to take necessary actions by changing the notification content.

In this modification example, the target position information may be separately recorded in the memory for the first examination and the second examination. Since the main purpose of the first examination is to determine the presence or absence of the lesion region, the endoscope 1 is pulled out over a relatively long period of time. On the other hand, since the main purpose of the second examination is a treatment such as excision of the lesion region, the endoscope 1 is pulled out in a relatively short period of time after the treatment is completed. Therefore, by setting the target position information suitable for each of the first examination and the second examination, it is possible to perform the notification at an appropriate timing. Similarly, in a case where the target position information is shared between the first examination and the second examination and the time until the notification is performed in a case where a notification condition is satisfied is changed between the first examination and the second examination, the notification can be made at an appropriate timing.

### Preferred Embodiment V: Determination of Insertion State

In a state in which the endoscope 1 is inserted into the subject (particularly, the insertion step), various situations may occur. For example, in a first specific state in which the distal end of the insertion part 10 is near the inner wall of the organ, a red ball phenomenon occurs in which the captured image becomes reddish as a whole. In addition, in a second specific state in which the distal end of the insertion part 10 is contaminated with the attachment, at least a part of the imaging range of the endoscope 1 is covered. In addition, as a result of the intestinal wall of the large intestine being crushed on the back side of the distal end of the insertion part 10, the distal end of the insertion part 10 may cause a third specific state in which the downstream side of the movement path 10X cannot be imaged. In addition, the insertion part 10 may be in a fourth specific state in which the deflection or the loop is formed. Each of the first specific state, the second specific state, and the third specific state constitutes a state of the distal end of the endoscope 1, and constitutes a specific state.

In the preferred embodiment V, the processor 8P determines the insertion state of the endoscope 1 into the subject on the basis of the captured image captured by the endoscope 1, and the distance information (the first distance, the specific insertion length, or the pulling-out length). The determination of the insertion state of the endoscope 1 means determining in what kind of situation the insertion part 10 is placed in the subject. The situation that may affect the endoscopy includes a situation in which the inner wall of the organ is stretched, a situation in which observation cannot be sufficiently performed, a situation in which the movement along the movement path is difficult, a situation in which the insertion is made more than necessary (formation of deflection or loop), and the like. Hereinafter, the determination method of the insertion state will be described in detail.

### (Determination of Insertion State (Organ Stretch State) in Which the Inner Wall of the Organ is Excessively Stretched)

The processor 8P determines whether or not the state of the distal end of the endoscope 1 is the first specific state on the basis of the captured image captured by the endoscope 1, and determines whether or not the insertion state of the endoscope 1 into the subject is the organ stretch state on the basis of the determination result thereof, and the change amount of the distance information (the first distance, the specific insertion length, or the pulling-out length).

In a case where the first specific state in which the distal end of the insertion part 10 is near the inner wall of the organ is continued despite the increase in the first distance or the specific insertion length, it can be determined that the distal end of the endoscope 1 continues to be pushed to the inner wall of the organ and the inner wall is stretched. Since the captured image is reddish as a whole in the first specific state, it is possible to determine the presence or absence of the occurrence of the first specific state by analyzing the captured image. For example, the processor 8P determines whether or not the first specific state has occurred from the output of an image recognition model obtained by inputting the captured image into the image recognition model generated by machine learning or the like. The processor 8P may determine whether or not the first specific state has occurred on the basis of the size of a red region included in the captured image, pattern matching with a reference captured image obtained in the first specific state, or the like. In a case where the determination result that the state is the first specific state is continuously obtained (continuously for a predetermined period of time), and the change amount (increase amount) of the distance information in the period in which the determination result is obtained is equal to or greater than the first threshold value, the processor 8P determines that the organ stretch state has occurred.

In a case where it is determined that the organ stretch state has occurred, it is preferable that the processor 8P outputs the operation support information based on the organ stretch state. For example, the processor 8P may display an alert indicating that the inner wall of the organ is stretched on the display device 7 or output the alert from the speaker. In addition, it is preferable that the processor 8P outputs, in addition to the output of this alert, a recommended operation (pulling toward, jiggling, or the like) not to further stretch the inner wall. This makes it possible to efficiently perform the insertion of the endoscope 1 while reducing the load applied to the subject.

Even in a case where the determination result that the state is the first specific state is continuously obtained (continuously for a predetermined period of time), and the change amount (increase amount) of the distance information in the period in which the determination result is obtained is equal to or greater than the first threshold value, the processor 8P may determine whether or not the state is the organ stretch state depending on the size of the distance information derived in the period.

For example, in a case where the determination result that the state is the first specific state is continuously obtained, and the change amount (increase amount) of the distance information in the period in which the determination result is obtained is equal to or greater than the first threshold value, the processor 8P determines a site corresponding to the minimum value or the maximum value of the distance information derived in the period on the basis of the first table data illustrated in FIG. 15. In a case where the determined site belongs to a range from the anus to the SDJ, the processor 8P determines that the state is the organ stretch state. In a case where the determined site is the splenic flexure, the processor 8P determines that the distal end of the endoscope 1 has reached the splenic flexure instead of determining the organ stretch state, and performs notification for prompting, for example, an angle operation. In a case where the determined site is the ileocecum, the processor 8P determines that the distal end of the endoscope 1 has reached the ileocecum instead of determining the organ stretch state, and performs control of giving a notification of, for example, the reaching of ileocecum. In this manner, it is possible to determine whether or not the state is the organ stretch state with higher accuracy.

### (Determination of Insertion State That May Result in Insufficient Observation (Insufficient Observation State))

The processor 8P determines whether or not the state of the distal end of the endoscope 1 is the second specific state on the basis of the captured image captured by the endoscope 1, and determines whether or not the insertion state of the endoscope 1 into the subject is the insufficient observation state on the basis of the determination result thereof, and the change amount of the distance information (the first distance, the specific insertion length, or the pulling-out length).

In a case where the first distance or the specific insertion length is increased even though the second specific state in which the distal end of the insertion part 10 is contaminated with the attachment is continued, the observation of the interior wall of the organ may not be sufficiently performed. In the second specific state, a shadow or reflected light generated by the attachment on the distal end of the insertion part 10 is included in the captured image. Therefore, it is possible to determine the presence or absence of the occurrence of the second specific state by analyzing the captured image. For example, the processor 8P determines whether or not the second specific state has occurred from the output of an image recognition model obtained by inputting the captured image into the image recognition model generated by machine learning or the like. The processor 8P may determine whether or not the second specific state has occurred on the basis of the size of a covered region included in the captured image. In a case where the determination result that the state is the second specific state is continuously obtained (continuously for a predetermined period of time), and the change amount (increase amount or decrease amount) of the distance information in the period in which the determination result is obtained is equal to or greater than the first threshold value, the processor 8P determines that the insufficient observation state has occurred.

In a case where it is determined that the insufficient observation state has occurred, it is preferable that the processor 8P outputs the operation support information based on the insufficient observation state. For example, the processor 8P may display a recommended operation (air supply, water supply, suction, or the like) for resolving the insufficient observation state (ensuring the visual field of the endoscope 1), on the display device 7, or output the recommended operation from the speaker. In this manner, it is possible to improve the quality of the endoscopy by preventing the overlooking of the lesion region.

The first threshold value used for this determination may be different between the insertion step and the pulling-out step. In general, in the pulling-out step, the observation is performed in more detail than in the insertion step. Therefore, for example, by setting the first threshold value in the pulling-out step to be smaller than the first threshold value in the insertion step, in the pulling-out step, the operation support information can be output only in a case where the second specific state is continued for a short period of time. Thereby, it is possible to improve the quality of the endoscopy.

The organ stretch state and the insufficient observation state described above constitute a first state.

### (Determination of Insertion State in Which Movement in Traveling Direction is Difficult (Insertion Difficulty State))

The processor 8P determines whether or not the state of the distal end of the endoscope 1 is the third specific state on the basis of the captured image captured by the endoscope 1, and determines whether or not the insertion state of the endoscope 1 into the subject is the insertion difficulty state on the basis of the determination result thereof, and the change amount of the distance information (the first distance, the specific insertion length, or the pulling-out length).

In a case where the third specific state in which the downstream side of the movement path 10X cannot be imaged by the distal end of the insertion part 10 is continued and the first distance or the specific insertion length is not changed, it can be said that the operator cannot determine the traveling direction of the endoscope 1. Since the direction in which the endoscope 1 is inserted (the downstream side of the movement path 10X) cannot be seen on the captured image in the third specific state, it is possible to determine the presence or absence of the occurrence of the third specific state by analyzing the captured image. For example, the processor 8P determines whether or not the third specific state has occurred from the output of an image recognition model obtained by inputting the captured image into the image recognition model generated by machine learning or the like. The processor 8P may determine whether or not the third specific state has occurred by determining whether or not a circular region corresponding to the shape of the lumen is included in the captured image. In a case where the determination result that the state is the third specific state is continuously obtained (continuously for a predetermined period of time), and the change amount (increase amount or decrease amount) of the distance information in the period in which the determination result is obtained is equal to or less than the second threshold value, the processor 8P determines that the insertion difficulty state has occurred.

In a case where it is determined that the insertion difficulty state has occurred, it is preferable that the processor 8P outputs the operation support information based on the insertion difficulty state. For example, the processor 8P may display a recommended operation (water supply operation, jiggling, or the like) for the progress of the insertion on the display device 7 or output the recommended operation from the speaker. This makes it possible to efficiently perform the insertion of the endoscope 1 while reducing the load applied to the subject.

### (Determination of Fourth Specific State (Formation of Deflection or Loop)

The processor 8P determines whether or not the insertion state of the endoscope 1 into the subject is the fourth specific state on the basis of the captured image captured by the endoscope 1, and the distance information (the first distance, the specific insertion length, or the pulling-out length). More specifically, the processor 8P performs the above-described reaching site recognition processing on the basis of the captured image captured by the endoscope 1, and determines the presence or absence of the occurrence of the fourth specific state on the basis of the result of the reaching site recognition processing and the distance information.

In a case where the distance information (referred to as distance LY1) is acquired at a first timing, the processor 8P recognizes the reaching site of the distal end of the endoscope 1 at the point in time when the distance information is acquired, by the reaching site recognition processing. In a case where the reaching site of the distal end of the endoscope 1 is recognized, the processor 8P acquires the distance information (referred to as distance LY2) corresponding to the reaching site, from the first table data exemplified in FIG. 15. In a case where the distance LY1 and the distance LY2 have substantially the same value, it can be said that the endoscope 1 is almost ideally inserted according to the reference data. On the other hand, in a case where the distance LY1 is greater than the distance LY2 by the threshold value or more, it can be determined that the endoscope 1 is inserted more than necessary, that is, the deflection or loop is formed. Therefore, the processor 8P compares the distance LY1 with the distance LY2, determines that the insertion state of the endoscope 1 is the fourth specific state in a case where the distance LY1 is greater than the distance LY2 by the threshold value or more, and determines that the insertion state of the endoscope 1 is not the fourth specific state in a case where the distance LY1 is not greater than the distance LY2 by the threshold value or more.

Alternatively, in a case where the distance information (referred to as distance LY1) is acquired at the first timing, the processor 8P acquires the reaching site (referred to as site J1) corresponding to the distance LY1, from the first table data exemplified in FIG. 15. In addition, the processor 8P compares the site JI with the past reaching site recognized by the reaching site recognition processing performed before the point in time when the distance LY1 is acquired, and determines that the state is the fourth specific state in a case where the site JI is not included in the past reaching site.

In a case where it is determined that the fourth specific state has occurred, it is preferable that the processor 8P notify of the estimated deflection/loop formation position or recommended operations (for example, manual compression, right twisting) to resolve the deflection/loop.
For example, the processor 8P can statistically estimate a site where the deflection or loop is likely to occur between the anus and the reaching site recognized by the reaching site recognition processing, from the reaching site.

The determination of the insertion state exemplified above may be performed only in the insertion step of the insertion step and the pulling-out step. In addition, which insertion state is to be determined may be determined for each reachable range of the distal end of the endoscope 1. For example, the determination of the presence or absence of the occurrence of the insufficient observation state may be performed in all (entire range from the anus to the ileocecum) of the insertion step and the pulling-out step, and the determination of the presence or absence of the occurrence of the organ stretch state, the insertion difficulty state, and the fourth specific state may be performed only in a range from the sigmoid colon to the splenic flexure. In addition, the insertion state to be determined may be changed between the insertion step and the pulling-out step. For example, in the insertion step, the presence or absence of the occurrence of the organ stretch state, the insufficient observation state, the insertion difficulty state, and the fourth specific state may be determined, and in the pulling-out step, the presence or absence of the occurrence of the organ stretch state and the insufficient observation state may be determined. In this manner, it is possible to reduce the processing load of the processor 8P.

### (Recording of Determination Result of Insertion State)

It is preferable that the processor 8P performs control to record, as the examination data, the determination result of the insertion state described above (the determination result that the state is the organ stretch state, the determination result that the state is the insufficient observation state, the determination result that the state is the insertion difficulty state, or the determination result that the state is the fourth specific state) in association with the elapsed time when the determination result is obtained. In this manner, for example, in the graph illustrated in FIG. 14, a period in which the state is the organ stretch state, a period in which the state is the insufficient observation state, a period in which the state is the insertion difficulty state, and a period in which the state is the fourth specific state (formation of the deflection or loop) can be checked together, which can be used for the evaluation of the procedure.

As described above, the detection unit 40 can also be integrally configured with an insertion assisting member of the endoscope 1. For example, the detection unit 40 may be integrally formed with the insertion assisting member to be inserted into the anus, or may be integrally formed with a mouthpiece-type insertion assisting member that is held in the mouth. In addition, the detection unit 40 may be integrally formed with the pants for endoscopy, or may be configured to be attachable to and detachable from the pants for endoscopy.

The technology of the present disclosure is not limited to the above description, and can be appropriately changed as described below.

For example, the endoscope 1 may be inserted into the body through the mouth or the nose of the subject 50. In this case, the detection unit 40 need only have a shape to be attachable to the mouth or the nose of the subject 50.

The tubular member 17 has the configuration in which the first member 14 and the second member 15 are provided, and each of the first member 14 and the second member 15 contains the magnetizable austenitic stainless steel, but one of the first member 14 or the second member 15 may be made of a non-magnetizable material. That is, the magnetic pattern may not be formed on one of the first member 14 or the second member 15. Even in such a case, since the magnetic flux densities BX, BY, and BZ described above can be detected from the tubular member 17, it is possible to determine the movement state and the rotation state of the insertion part 10.

In the above description, in the tubular member 17, the two types of magnetic pole regions are alternately arranged in the longitudinal direction to form the magnetic pattern, and the movement state of the insertion part 10 in the longitudinal direction is determined on the basis of the combination of the classification levels of the magnetic information in the two directions detected from the magnetic pattern. However, the two types of magnetic pole regions formed on the tubular member 17 may not be alternately arranged in the longitudinal direction. Even in such a case, the movement state of the insertion part 10 in the longitudinal direction can be determined on the basis of the combination of the classification levels of the magnetic information in the two directions detected from the magnetic pattern.

In addition, as a modification example, the movement state of the insertion part 10 in the longitudinal direction may be determined by forming a pattern more complicated than the magnetic pattern described above on the tubular member 17 and detecting the pattern by the magnetic detection units 43 and 44. Specifically, a table in which each position of the tubular member 17 in the longitudinal direction and the magnetic flux density BX or the magnetic flux density BY (classification level) detected at each position are associated with each other may be recorded in a memory, and the processor 8P may classify the magnetic flux density BX or the magnetic flux density BY detected by the magnetic detection unit 43 to acquire the classification level, and may acquire the information on the position corresponding to the classification level from the table to determine the insertion length of the insertion part 10. As a result, the insertion length of the insertion part 10 can be finely determined. In addition, the magnetic detection units 43 and 44 can detect the magnetic flux densities in one direction, so that the cost can be reduced.

As described above, at least the following matters are described in the present specification.
(1)
A processing device comprising:
a processor configured to:
   acquire a distance from a reference position on a movement path of an endoscope to a distal end of the endoscope that is moved along the movement path;
   acquire target position information indicating a target position on the movement path from a memory; and
   perform control of giving a notification based on the distance and the target position information.
(2)
The processing device according to (1),
wherein the target position information includes information on the distance acquired in a state where, in a past examination performed using an endoscope, the endoscope images a region-of-interest in a subject.
(3)
The processing device according to (2),
wherein the reference position is an ending point position of the movement path, and
the target position information is acquired in a period in which the endoscope is moved from the ending point position toward a starting point of the movement path in the past examination.
(4)
The processing device according to (1),
wherein the processor is configured to:
   store the distance acquired in a first period in which the endoscope is moving from a starting point to an ending point of the movement path and in a state in which the endoscope is imaging a region-of-interest in a subject, in the memory as the target position information; and
   perform the control in a second period in which the endoscope is moving from the ending point to the starting point of the movement path.
(5)
The processing device according to (4),
wherein the processor is configured to perform a control of giving a notification based on the distance acquired in the second period and a predetermined range of the movement path including the target position information.
(6)
The processing device according to (1),
wherein the processor is configured to perform a control of giving a notification of a presence of a region to be noticed in a subject into which the endoscope is inserted, based on the distance and the target position information.
(7)
The processing device according to (6),
wherein the processor is configured to:
   perform detection processing of acquiring an image captured by the endoscope and detecting a region to be noticed in the subject based on the image; and
   perform the control based on a result of the detection processing, the distance, and the target position information.
(8)
The processing device according to (1),
wherein the target position information includes information on the distance that is acquirable in a state in which a distal end of the endoscope is at a position on a starting point side of the movement path.
(9)
The processing device according to (8),
wherein the processor is configured to determine a movement direction of the endoscope and perform the control based on the distance, the target position information, and the movement direction of the endoscope.
(10)
The processing device according to (9),
wherein the processor is configured to, in a case where the movement direction is a direction from an ending point to a starting point of the movement path, perform a control of giving a notification based on the distance and the target position information.
(11)
The processing device according to (9) or (10),
wherein the processor is configured to determine the movement direction based on a time change of the distance.
(12)
The processing device according to any of (1) to (11),
wherein the processor is configured to perform a control of giving a notification of information related to an end of an examination using the endoscope based on the distance and the target position information.
(13)
The processing device according to (12),
wherein the processor is configured to:
   perform detection processing of acquiring an image captured by the endoscope and detecting a region to be noticed in a subject based on the image; and
   change a notification content of the information in a case where the region is detected by the detection processing after the control is performed.
(14)
The processing device according to any of (1) to (13),
wherein a magnetic pattern is formed along a longitudinal direction on an insertion part of the endoscope, and
the processor acquires the distance based on a magnetic field from the magnetic pattern, which is detected by a magnetic detection unit installed outside a subject to which the endoscope is inserted.
(15)
An endoscope device comprising: the processing device according to any of (1) to (14); and the endoscope.
(16)
The endoscope device according to (15), further comprising:
a magnetic detection unit disposed on the movement path,
wherein an insertion part of the endoscope has a member containing metal, which extends in a longitudinal direction and has a magnetic pattern integrally formed along the longitudinal direction,
the magnetic detection unit detects a magnetic field from the member, and
the processor derives the distance based on the magnetic field detected by the magnetic detection unit.
(17)
The endoscope device according to (16),
wherein the insertion part includes a soft portion of the endoscope.
(18)
The endoscope device according to (17),
wherein the soft portion has a cylindrical member having an insulating property, a cylindrical first member that contains metal and is inserted into the cylindrical member, and a cylindrical second member that contains metal and is inserted into the first member, and
the member includes at least one of the first member or the second member.
(19)
The endoscope device according to (18),
wherein at least one of the first member or the second member is made of magnetizable austenitic stainless steel.
(20)
The endoscope device according to (15), further comprising:
a magnetic detection unit disposed on the movement path,
wherein an insertion part of the endoscope has a member containing metal, which extends in a longitudinal direction and has a magnetic pattern formed along the longitudinal direction,
the magnetic detection unit detects a magnetic field from the member,
the processor derives the distance based on the magnetic field detected by the magnetic detection unit,
the insertion part has a cylindrical member having an insulating property, a cylindrical first member that contains metal and is inserted into the cylindrical member, and a cylindrical second member that contains metal and is inserted into the first member,
the member includes at least one of the first member or the second member,
the first member is a spiral tube, and
the second member is a net body.
(21)
The endoscope device according to any of (16) to (20),
wherein the magnetic detection unit detects a first magnetic flux density in a first direction and a second magnetic flux density in a second direction intersecting the first direction, at a plurality of positions along the longitudinal direction of the member.
(22)
A processing method comprising:
acquiring a distance from a reference position on a movement path of an endoscope to a distal end of the endoscope that is moved along the movement path;
acquiring target position information indicating a target position on the movement path from a memory; and
performing control of giving a notification based on the distance and the target position information.

Although various embodiments have been described above, it goes without saying that the present invention is not limited to these examples. It is apparent that those skilled in the art may perceive various modification examples or correction examples within the scope disclosed in the claims, and those examples are also understood as falling within the technical scope of the present invention. In addition, each constituent in the embodiment may be used in any combination without departing from the gist of the invention.

The present application is based on Japanese Patent Application (JP2022-174967) filed on Oct. 31, 2022, the content of which is incorporated in the present application by reference.

### EXPLANATION OF REFERENCES

1: endoscope
MA, MA1, MA2: magnetic pole portion
4P: processor
4: processor device
5: light source device
6: input unit
7: display device
8: expansion device
8P: processor
10A: soft portion
10B: bendable part
10C: distal end part
10: insertion part
11: operating part
12: angle knob
13A, 13B: connector portion
13: universal cord
14: first member
15a: metal strip
15: second member
16A, 16B: cap
17N: positive pole region
17S: negative pole region
17: tubular member
18A: inner resin layer
18B: outer resin layer
18: outer skin layer
19: coating layer
40: detection unit
42: housing
42A: body part
42B: lid portion
42a: flat plate portion
42b: side wall portion
42c: inner wall portion
41A, 41B, 41: through-hole
43, 44: magnetic detection unit (magnetic detector)
45: communication chip
46: storage battery
47: power receiving coil
50A: anus
53: rectum
54: sigmoid colon
55: descending colon
56: transverse colon
57: ascending colon
58: ileocecum
50: subject
100: endoscope device
200: endoscope system
300: magnetic field generation device
PO1, PO2: position

What is claimed is:

1. An endoscope device comprising:
an endoscope;
a processing device comprising a processor configured to:
acquire a distance from a reference position on a movement path of the endoscope to a distal end of the endoscope that is moved along the movement path;
acquire, from a memory, target position information indicating a target position on the movement path; and
perform control of giving a notification based on the distance and the target position information; and
a magnetic detector disposed on the movement path,
wherein an insertion part of the endoscope has a member containing metal, which extends in a longitudinal direction and has a magnetic pattern integrally formed along the longitudinal direction,
the magnetic detector detects a magnetic field from the member,
the processor is configured to derive the distance based on the magnetic field detected by the magnetic detector,
the insertion part includes a soft portion of the endoscope,
the soft portion has a cylindrical member having an insulating property, a cylindrical first member that contains metal and is inserted into the cylindrical member, and a cylindrical second member that contains metal and is inserted into the first member, and
the member containing metal includes at least one of the first member or the second member.

2. The endoscope device according to claim 1,
wherein the target position information includes information on the distance acquired in a state where, in a past examination performed using an endoscope, the endoscope images a region-of-interest in a subject under the past examination.

3. The endoscope device according to claim 2,
wherein the reference position is a position of an ending point of the movement path, and
the target position information is acquired in a period in which the endoscope is moved from the position of the ending point toward a starting point of the movement path in the past examination.

4. The endoscope device according to claim 1,
wherein the processor is configured to:
store the distance acquired in a first period in which the endoscope is moving from a starting point to an ending point of the movement path and in a state in which the endoscope is imaging a region-of-interest in a subject, in the memory as the target position information; and
perform the control in a second period in which the endoscope is moving from the ending point to the starting point of the movement path.

5. The endoscope device according to claim 4,
wherein the processor is configured to perform a control of giving a notification based on the distance acquired in the second period and a predetermined range of the movement path including the target position information.

6. The endoscope device according to claim 1,
wherein the processor is configured to perform a control of giving a notification of a presence of a region to be noticed in a subject into which the endoscope is inserted, based on the distance and the target position information.

7. The endoscope device according to claim 6,
wherein the processor is configured to:
perform detection processing of acquiring an image captured by the endoscope and detecting a region to be noticed in the subject based on the image; and
perform the control based on a result of the detection processing, the distance, and the target position information.

8. The endoscope device according to claim 1,
wherein the processor is configured to determine a movement direction of the endoscope and perform the control based on the distance, the target position information, and the movement direction of the endoscope.

9. The endoscope device according to claim 8,
wherein the processor is configured to, in a case where the movement direction is a direction from an ending point to a starting point of the movement path, perform a control of giving a notification based on the distance and the target position information.

10. The endoscope device according to claim 8,
wherein the processor is configured to determine the movement direction based on a time change of the distance.

11. The endoscope device according to claim 1,
wherein the processor is configured to perform a control of giving a notification of information related to an end of an examination using the endoscope based on the distance and the target position information.

12. The endoscope device according to claim 11,
wherein the processor is configured to:
perform detection processing of acquiring an image captured by the endoscope and detecting a region to be noticed in a subject based on the image; and
change a content of the notification of information in a case where the region is detected by the detection processing after the control is performed.

13. The endoscope device according to claim 1,
wherein a magnetic pattern is formed along a longitudinal direction on an insertion part of the endoscope, and
the processor is configured to acquire the distance based on a magnetic field from the magnetic pattern, which is detected by a magnetic detector provided outside a subject to which the endoscope is inserted.

14. The endoscope device according to claim 1,
wherein at least one of the first member or the second member is made from magnetizable austenitic stainless steel.

15. The endoscope device according to claim 1, wherein
the first member is a spiral tube, and
the second member is a net body.

16. The endoscope device according to claim 1,
wherein the magnetic detector detects a first magnetic flux density in a first direction and a second magnetic flux density in a second direction intersecting the first direction, at a plurality of positions along the longitudinal direction of the member containing metal.
